(19) United States
(12) Patent Application Publication
(10) Pub. No.: US 2024/0110231 A1
(43) Pub. Date: Apr. 4, 2024

(54) RNA TAGGING SYSTEM FOR VISUALIZATION OF SINGLE MRNA MOLECULES

(71) Applicant: Albert Einstein College of Medicine, Bronx, NY (US)

(72) Inventors: Robert H. Singer, New York, NY (US); Evelina Tutucci, New York, NY (US); Maria Vera Ugalde, New York, NY (US)

(21) Appl. No.: 18/242,217

(22) Filed: Sep. 5, 2023

Related U.S. Application Data
(62) Division of application No. 16/606,046, filed on Oct. 17, 2019, now Pat. No. 11,781,173.
(60) Provisional application No. 62/487,058, filed on Apr. 19, 2017.

(51) Int. Cl. C12Q 1/6841; C12Q 1/6897

(52) U.S. Cl. CPC C12Q 1/6841; C12Q 1/6897

(57) ABSTRACT

An RNA tagging system for visualization of single mRNA molecules based on a MSB-MCP system, as well as methods of use.

### CROSS-REFERENCE TO RELATED APPLICATIONS

This application is a Divisional application of U.S. application Ser. No. 16/606,046, filed on Oct. 17, 2019, which is a U.S. National Stage Entry under 35 U.S.C. § 371 of PCT International Patent Application No. PCT/US2018/028269, filed Apr. 19, 2018, which claims benefit of U.S. Provisional Application No. 62/487,058, filed Apr. 19, 2017, the contents of each of which are incorporated herein by reference into the subject application.

### STATEMENT OF GOVERNMENT SUPPORT

This invention was made with government support under grant number GM57071 awarded by the National Institutes of Health. The government has certain rights in the invention.

### REFERENCE TO SEQUENCE LISTING

This application contains a Sequence Listing that has been submitted electronically as an XML file named AET-02802_SL.xml. The XML file, created on Nov. 28, 2023, is 68,124 bytes in size. The material in the XML file is hereby incorporated by reference.

### BACKGROUND OF THE INVENTION

Throughout this application various publications are referred to. Full citations for the references may be found at the end of the specification. The disclosures of these publications are hereby incorporated by reference in their entirety into the subject application to more fully describe the art to which the subject invention pertains.

The ability to visualize single molecules in intact cells is a powerful tool to study gene expression quantitatively from transcription to translation with high temporal and spatial resolution (Vera et al., 2016). However, the last step of the mRNA life cycle, degradation, remained a challenging event to visualize in living cells at the single molecule level. Previous work from our lab used single molecule in situ hybridization (smFISH) to analyze the degradation of highly unstable mRNAs in yeast (Trcek et al., 2011). This work provided the first evidence that the promoter sequence of cell cycle regulated genes dictates when mRNAs decay in the cytoplasm. However, using fixed cells precludes determining single mRNA dynamics from transcription to degradation in a single cell. Hence, important information is missed about where mRNA degradation occurs in the cell and how variable this process is from cell to cell.

To report mRNA decay in living cells we used the available MS2-MCP system. This method utilizes RNA loops derived from the single-stranded RNA bacteriophage MS2. In the phage genome, the MS2 binding sites (MBS) and the MS2 coat protein homo-dimer (MCP) interact to control viral particle assembly (Bernardi and Spahr, 1972). For mRNA detection, 24 MBSs are inserted in the 3′UTR of an mRNA of interest and co-expression of MCP fused with fluorescent proteins renders single mRNAs visible using wide-field epi-fluorescence microscopy. This approach has been used to image and follow single mRNA molecules in living eukaryotic cells to study mRNA transcription, export, localization and translation (Bertrand et al., 1998; Fusco et al., 2003; Grunwald and Singer, 2010; Larson et al., 2011; Shav-Tal et al., 2004; Wu et al., 2016). However, the attempt to use the available MBS-MCP system to study mRNA decay of tagged mRNAs in S. cerevisiae revealed that mRNA degradation is impaired by MCP binding to MBS, which can inhibit the cytoplasmic exonuclease Xml (Garcia and Parker, 2015, 2016). Consequently, a significant fraction of the signal observed using the MBS-MCP system is due to 3′ decay fragments containing MS2 loops (Garcia and Parker, 2015; Heinrich et al., 2017). This degradation inhibition differs based on mRNA levels, whether the mRNA is expressed from a plasmid or from the endogenous locus and the intrinsic stability of the mRNA (Haimovich et al., 2016) and is enhanced in stress conditions, such as glucose starvation (Heinrich et al., 2017). Because the MBS system has been used to study various aspects of cytoplasmic mRNA regulation in living yeast (Sheth and Parker, 2003; Zid and O'Shea, 2014; Zipor et al., 2009), the uncertainty as to whether the MBS signal represents full-length mRNA raises the concern that the available MBS-MCP system can yield spurious results in S. cerevisiae.

The present invention addresses the need for a new method and system for visualization of single mRNA molecules.

### SUMMARY OF THE INVENTION

A nucleic acid is provided encoding from twelve to twenty four loops of 5′-ANC/UA-3′, wherein the 5′ end of each loop is connected to a sequence of eight nucleotides, seven of which are complementary to a sequence of seven nucleotides connected to the 3′ end of the same loop, such that a stem and loop structure is formed for each loop, and wherein a stem of each loop is separated from a stem of each adjacent loop by a nucleotide sequence of more than 39 nucleotides.

A nucleic acid is provided encoding from seven to twelve loops of 5′-ANC/UA-3′, wherein the 5′ end of each loop is connected to a sequence of eight nucleotides, seven of which are complementary to a sequence of seven nucleotides connected to the 3′ end of the same loop, such that a stem and loop structure is formed for each loop, and wherein a stem of each loop is separated from a stem of each adjacent loop by a nucleotide sequence of more than 40 nucleotides.

In an embodiment, the nucleic acid comprises SEQ ID NO:2 or 3. In an embodiment, the nucleic acid comprises SEQ ID NO:2. In an embodiment, the nucleic acid comprises SEQ ID NO:3.

In an embodiment, the nucleic acid encodes from twelve to twenty four loops. In an embodiment, the nucleic acid encodes twelve loops. In an embodiment, the nucleic acid encodes twenty four loops.

Also provided is a nucleic acid encoding, in 5′ to 3′ order or 3′ to 5′ order:
(i) a CYC1 promoter;
(ii) a MS2 bacteriophage coat protein homo-dimer (MCP);
(iii) a first fluorescent protein;
(iv) a second fluorescent protein;
(v) a nuclear localization sequence (NLS);
(vi) a CYC1 terminator sequence.

Also provided is a nucleic acid encoding, in 5′ to 3′ order or 3′ to 5′ order:
(i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence.

In an embodiment, the nucleic acid comprises SEQ ID NO:4 or 32. In an embodiment, the nucleic acid comprises SEQ ID NO:4. In an embodiment, the nucleic acid comprises SEQ ID NO:32.

A kit is provided comprising two plasmids, (1) and (2), wherein:
(1) encodes from seven to twenty four loops of 5′-ANC/UA-3′, each wherein the 5′ end of each loop is connected to a sequence of eight nucleotides, seven of which nucleotides complementary to a sequence of seven nucleotides connected to the 3′ end of the same loop, such that a stem and loop structure is formed for each loop, and wherein each loop is separated from each adjacent loop by a nucleotide sequence of 40-55 nucleotides, and
(2) encodes, in 5′ to 3′ order or 3′ to 5′ order:
(A) (i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence, or
(B) (i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence; and
(3) instructions for use in visualizing an RNA of interest in a eukaryotic cell.

A kit is provided comprising two plasmids, (i) and (ii), wherein:
(i) encodes from seven to twelve loops of 5′-ANC/UA-3′, each wherein the 5′ end of each loop is connected to a sequence of eight nucleotides, seven of which nucleotides complementary to a sequence of seven nucleotides connected to the 3′ end of the same loop, such that a stem and loop structure is formed for each loop, and wherein each loop is separated from each adjacent loop by a nucleotide sequence of 45-55 nucleotides, and
(ii) encodes, in 5′ to 3′ order or 3′ to 5′ order:
(i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence, and
(iii) instructions for use in visualizing an RNA of interest in a eukaryotic cell.

Also provided is a method of detecting or of monitoring an RNA of interest in a yeast cell, the method comprising:
(i) inserting in a cell the nucleic acid, encoding from twelve to twenty four loops of 5′-ANC/UA-3′, as described herein into the 3′-UTR of a gene encoding the RNA of interest by homologous recombination and using Cre-Lox recombination so as to remove a marker gene so as to tag the RNA of interest with the twelve to twenty four loops;
(ii) transfecting the cell with a nucleic acid, encoding an MCP, as described herein so as to permit expression of a fusion protein comprising the MCP, NLS and the first and second fluorescent proteins
(iii) detecting or monitoring the movement and/or location of a fluorescent signal of the first and second fluorescent proteins so as to detect or monitor the RNA of interest.

Also provided is a method of detecting or of monitoring an RNA of interest in a yeast cell, the method comprising:
(i) inserting in a cell the nucleic acid, encoding from seven to twelve four loops of 5′-ANC/UA-3′, as described herein into the 3′-UTR of a gene encoding the RNA of interest by homologous recombination and using Cre-Lox recombination so as to remove a marker gene so as to tag the RNA of interest with the seven to twelve four loops;
(ii) transfecting the cell with a nucleic acid, encoding an MCP, as described herein so as to permit expression of a fusion protein comprising the MCP, NLS and the first and second fluorescent proteins
(iii) detecting or monitoring the movement and/or location of a fluorescent signal of the first and second fluorescent proteins so as to detect or monitor the RNA of interest.

Also provided is a method of detecting or of monitoring an RNA of interest in a yeast cell, the method comprising:
(i) inserting in a cell the nucleic acid, encoding from twelve to twenty four loops of 5′-ANC/UA-3′, as described herein into the 3′-UTR of a gene encoding the RNA of interest by homologous recombination and using Cre-Lox recombination so as to remove a marker gene so as to tag the RNA of interest with the twelve to twenty four loops;
(ii) transfecting the cell with a nucleic acid, encoding an MCP, as described herein so as to permit expression of a fusion protein comprising the MCP, NLS and the first and second fluorescent proteins and second fluorescent proteins so as to detect or monitor the RNA of interest.

Also provided is a method of detecting or of monitoring an RNA of interest in a yeast cell, the method comprising:
(i) inserting in a cell the nucleic acid, encoding from seven to twelve loops of 5′-ANC/UA-3′, as described herein into the 3′-UTR of a gene encoding the RNA of interest by homologous recombination and using Cre-Lox recombination so as to remove a marker gene so as to tag the RNA of interest with the seven to twelve loops;
(ii) transfecting the cell with a nucleic acid, encoding an MCP, as described herein so as to permit expression of a fusion protein comprising the MCP, NLS and the first and second fluorescent proteins
(iii) detecting or monitoring the movement and/or location of a fluorescent signal of the first and second fluorescent proteins so as to detect or monitor the RNA of interest.

Also provided is a method of detecting or of monitoring an RNA of interest in a eukaryotic cell, the method comprising:
(i) transfecting the eukaryotic cell with a plasmid or RNA that has been in vitro modified to encode from twelve to twenty four loops of 5′-ANC/UA-3′, as described herein, into the 3′-UTR of a gene encoding the RNA of interest
(ii) transfecting the cell with a nucleic acid encoding a MS2 bacteriophage coat protein homo-dimer (MCP) and a fluorescent protein;
(iii) detecting or monitoring the movement and/or location of a fluorescent signal of the first and second fluorescent proteins so as to detect or monitor the RNA of interest.

Also provided is a method of detecting or of monitoring an RNA of interest in a eukaryotic cell, the method comprising:
(i) transfecting the eukaryotic cell with a plasmid or RNA that has been in vitro modified to encode from seven to twelve loops of 5′-ANC/UA-3′, as described herein, into the 3′-UTR of a gene encoding the RNA of interest
(ii) transfecting the cell with a nucleic acid encoding a MS2 bacteriophage coat protein homo-dimer (MCP) and a fluorescent protein;
(iii) detecting or monitoring the movement and/or location of a fluorescent signal of the first and second fluorescent proteins so as to detect or monitor the RNA of interest.

Additional objects of the invention will be apparent from the description which follows.

### BRIEF DESCRIPTION OF THE DRAWINGS

FIG. 1A-1H: Current MBS systems resist degradation in yeast. (A and B) Scheme of ASH1 and MDN1 loci tagged currently used MBS (A) Schematic representation of 24×MBSV5 inserted in the 3′UTR of endogenous ASH1 gene. All loops have a different stem sequence to avoid recombination. Purple boxes represent the localization sequences of ASH1 mRNA. (B) Schematic representation of 24×MBSORF inserted in the 3′UTR of the endogenous MDN1 gene. Loops have no STOP codons in the 3 frames to avoid NMD. (A and B) Dotted lines designate smFISH probe positions recognizing the CDS (green) or MBS sequences (red). (C and D) Two color smFISH for tagged mRNAs (C) ASH1 mRNA tagged with 24×MBSV5 and (D) MDN1 tagged with 24×MBSORF, in cells expressing MCP (YcpLac111 CYC1p-MCP-NLS-2xyeGFP) or the vector alone (YcpLac111). (C) DIC/MERGE shows the overlap of the DAPI signal in the nucleus (blue), smFISH for the ASH1 CDS (green) and the MBS (red) with the differential interference contrast (DIC) image. (D) MERGE shows the overlap of the DAPI (blue), smFISH for the MDN1 CDS (green) and the MBS (red). Yellow lines define the shape of a single cell and the corresponding cell cycle stage. Arrows designate single mRNAs. Scale bar=5 μm. TS=transcription site. (E and F) Quantification of smFISH represented in 1C and 1D with CDS probes (green plots) or MBS probes (red) reported as frequency distribution of mature ASH1 (E) and MDN1 (F) mRNAs per cell. Mean and SD of two biological replicates, n=−500 cells per experiment. (G) MBS aggregates in the cytoplasm detected as bright fluorescent spots by smFISH. Merge shows the overlap of the DAPI (blue), smFISH for the CDS (green, for either ASH1 or MDN1) and the MBS (red, for MBSVS or MBSORF) in cells co-expressing MCP. MBS aggregates (arrows) are not detected with CDS probes. The percentage indicates cells positive for MBS aggregates. Scale bar=5 μm (H) MBS aggregates in live cells detected in the cytoplasm of living cells co-expressing MCP. Single ASH1 (left) and MDN1 (right) mRNAs are visualized as discrete GFP dots in the cytoplasm (white arrows). MBS aggregates are visualized as GFP dots brighter than single mRNAs (yellow arrows).

FIG. 2A-2K: Design and characterization of a new MBS-MCP system (A-C) Schematic representation of current MBS systems. (A) MBS wt sequence from the bacteriophage MS2. Nucleotide positions are relative to the translation start codon AUG (+1). FIG. 2A shows ACAUCACCAUUACCCAUCU (SEQ ID NO: 33). (B) MBSORF loops are dimers of 2 different loop sequences distanced by 20 nts repeated every 50 nts. MBSORF is C-variant (cytosine at position −5). The stem loop is 7 nts long. (C) MBSVS loops have non repeated sequences and 30 nt linkers with stem loops of 9 nts. All loops are C-variant. (D) RNA loop sequences used for EMSA assays. MBS is the original MS2 bacteriophage sequence (MBS shows ACAUGAGGAUCACCCAUGU (SEQ ID NO: 34) or ACAUGAGGAUUACCCAUGU (SEQ ID NO: 35). Loops 1 and 2 have randomized stem sequence of 7 nts (Loop1 shows GACGCAGGACCACCGCGUC (SEQ ID NO: 36) or GACGCAGGACUACCGCGUC (SEQ ID NO: 37) (Loop2: CGCAGAGGAACACCCUGCG (SEQ ID NO: 38) or CGCAGAGGAAUACCCUGCG (SEQ ID NO: 39). The three loops were generated either as a wt (U) or C variants (C). The affinity of MCP for the six loops was tested by EMSA. (E-G) Binding affinity of MCP for the MBS C-variants and wt-variants. Plot of the fraction of RNA bound as a function of MCP concentration and its fit to the Hill equation. The Kd from three independent measurements is indicated on the plots for (E) the original MBS sequence, (F) the Loop1 and (G) the Loop2, either wt or C-variant. (H and I) Schematic representation of new MBS systems. MBSV6 and MBSV7 have a randomized sequence of twelve stem loops and linkers. The length of the stem is 7 nts and the linkers are 50 nt (MBSV6, H) or 40 nts (MBSV7, I). MBSV6 and MBSV7 were synthetized in two versions: C-variant (12 loops with C) or a wt-variant (12 loops with U). (J-K) Image of living cells co-expressing MCP. (J) ASH1 24×MBSV6 C variant (left) or MDN1 24×MBSV7 C variant (right). (K) ASH1 24×MBSV6 wt-variant (left) or MDN1 24×MBSV7 wt-variant (right). Yellow arrows indicate MBS aggregates. White arrows indicate single mRNAs.

FIG. 3A-3H: Endogenous mRNAs tagged with the new MBS systems are rapidly degraded. (A and B) Two color smFISH for ASH1 mRNAs (A) and MDN1 mRNAs (B) tagged with 24×MBSV7, in cells expressing MCP (YcpLac111 CYC1p-MCP-NLS-2xyeGFP) or the vector alone (YcpLac111). (A) DIC/MERGE shows the overlap of the DAPI (blue), smFISH for the ASH1 CDS (green) and the MBSV7 (red) with the DIC image. (B) MERGE shows the overlap of the DAPI signal (blue), smFISH for the MDN1 CDS (green) and the MBSV7 (red). The shape of the cell is indicated and the corresponding cell cycle stage. Scale bar=5 μm. (C and D) Quantification of smFISH represented in 3A and 3B with CDS probes (green plots) or MBS probes (red) reported as frequency distribution of mature ASH1 (C) and MDN1 (D) mRNAs per cell. Mean and SD of two biological replicates, n=˜500 cells per experiment. (E and F) Two color smFISH for ASH1 mRNAs (E) and MDN1 mRNAs (F) tagged with 24×MBSV6, in cells expressing MCP (YcpLac111 CYC 1p-MCP-NLS-2xyeGFP) or the vector alone (YcpLac111). (A) DIC/MERGE shows the overlap of the DAPI (blue), smFISH for the ASH1 CDS (green) and the MBSV6 (red) with the DIC image. (B) MERGE shows the overlap of the DAPI signal (blue), smFISH for the MDN1 CDS (green) and the MBSV6 (red). The shape of the cell is indicated and the corresponding cell cycle stage. Scale bar=5 μm. (G and H) Quantification of smFISH represented in 3E and 3F with CDS probes (green plots) or MBSV6 probes (red) reported as frequency distribution of mature ASH1 (G) and MDN1 (H) mRNAs per cell. Mean and SD of two biological replicates, n=˜500 cells per experiment.

FIG. 4A-4D: Reducing the number of MBS for endogenous mRNAs tagging favors degradation. (A and B) Two color smFISH for ASH1 mRNAs (A) and MDN1 mRNAs (B) tagged with 12×MBS V6, in cells expressing MCP (YcpLac111 CYC1p-MCP-NLS-2xyeGFP) or the vector alone (YcpLac111). (A) DIC/MERGE shows the overlap of the DAPI (blue), smFISH for the ASH1 CDS (green) and the MBSV6 (red) with the DIC image. (B) MERGE shows the overlap of the DAPI signal (blue), smFISH for the MDN1 CDS (green) and the MBSV6 (red). The shape of the cell is indicated and the corresponding cell cycle stage. Scale bar=5 μm. (C and D) Quantification of smFISH represented in 4A and 4B with CDS probes (green plots) or MBSV6 probes (red) reported as frequency distribution of mature ASH1 (C) and MDN1 (D) mRNAs per cell. Mean and SD of two biological replicates, n=−500 cells per experiment.

FIG. 5A-5D: ASH1 mRNA expression throughout the cell cycle can be measured with MBSV6-MCP (A) Scheme of ASH1 mRNA expression during the cell cycle (marked on red arrow). Green dots represent ASH1 mRNA. mRubyTub1 (red) marks the spindle pole body (SPB), duplicated during S phase. The bud emergence (outlined) starts during S-phase and ends with the formation of the daughter cell. (B) Representative images of video. Simultaneous two-color imaging of cells co-expressing ASH1 24×MBSV6-MCP (gray) and mRubyTub1 (red). Time 0 indicates the beginning of anaphase. Images were acquired every 2 minutes. Single molecules are seen at 10 min and end at 24 min. (C) Quantification of single ASH1 mRNAs of cell shown in (B) during the cell cycle (black dots connected by green line). Black curve indicates cytoplasmic ASH1 mRNA profiles fitted to a single exponential decay model.t_{1/2}=5.6 min. (D) Quantification of single DOA1 mRNAs tagged with 24×MBSV6-MCP during the complete cell cycle (n=15). Images were acquired every 2 minutes.

### DETAILED DESCRIPTION OF THE INVENTION

To address this problem of spurious MBS-MCP system signals, the inventors designed a new MBS system that mimics the actual regulation of the endogenous untagged mRNA in eukaryotic cells.

A nucleic acid is provided encoding from twelve to twenty four loops of 5′-ANC/UA-3′, wherein the 5′ end of each loop is connected to a sequence of eight nucleotides, seven of which are complementary to a sequence of seven nucleotides connected to the 3′ end of the same loop, such that a stem and loop structure is formed for each loop, and wherein a stem of each loop is separated from a stem of each adjacent loop by a nucleotide sequence of more than 39 nucleotides.

A nucleic acid is provided encoding from seven to twelve loops of 5′-ANC/UA-3′, wherein the 5′ end of each loop is connected to a sequence of eight nucleotides, seven of which are complementary to a sequence of seven nucleotides connected to the 3′ end of the same loop, such that a stem and loop structure is formed for each loop, and wherein a stem of each loop is separated from a stem of each adjacent loop by a nucleotide sequence of more than 40 nucleotides.

In an embodiment, a stem of each loop is separated from a stem of each adjacent loop by a nucleotide sequence of 40-55 nucleotides. In an embodiment, a stem of each loop is separated from a stem of each adjacent loop by a nucleotide sequence of 41-60 nucleotides. In an embodiment, a stem of each loop is separated from a stem of each adjacent loop by a nucleotide sequence of 45-55 nucleotides. In an embodiment, a stem of each loop is separated from a stem of each adjacent loop by a nucleotide sequence of 50 nucleotides.

In an embodiment, the nucleotide of the eight nucleotides that is not complementary to the seven nucleotides connected to the 3′ end of the loop is an unpaired purine. In an embodiment, the unpaired purine is an A. In an embodiment, the nucleotide of the eight nucleotides that is not complementary to the seven nucleotides connected to the 3′ end of the loop is the third nucleotide of the sequence of eight nucleotides as counted from the 5′ end of the ‘-ANC/UA-3’ loop. In an embodiment, the first two nucleotides of the sequence of eight nucleotides as counted from the 5′ end of the ‘-ANC/UA-3’ loop are, respectively, complementary to the first two nucleotides of the sequence of seven nucleotides as counted from the 3′ end of the ‘-ANC/UA-3’ loop. In an embodiment, the fourth to eighth nucleotides of the sequence of eight nucleotides as counted from the 5′ end of the ‘-ANC/UA-3’ loop are, respectively, complementary to the third to seventh nucleotides, respectively, of the sequence of seven nucleotides as counted from the 3′ end of the ‘-ANC/UA-3’ loop.

In an embodiment, N in the sequence ANC/UA is any nucleotide or ribonucleotide. In an embodiment, N is A. In an embodiment, N is U. In an embodiment, N is C. In an embodiment, N is G.

In an embodiment, the nucleic acid, at a 3′ portion thereof, further comprises a two LoxP sites, optionally separated by a marker gene. In an embodiment, the marker gene is a kanamycin resistance gene.

In an embodiment, the nucleic acid encodes twelve loops of 5′-ANC/UA-3′.

In an embodiment, each loop stem is separated from each adjacent loop stem by a nucleotide sequence of 50 nucleotides.

In an embodiment, the stem of the stem and loop structure for each of the loops has a different sequence than the stems of the stem and loop structure for all of the remaining loops.

In an embodiment, the stem of the stem and loop structure for each of the loops has the same sequence than the stems of the stem and loop structure for all of the remaining loops.

In an embodiment, the loops encoded by the nucleic acid all have the sequence 5′-ANUA-3′.

In an embodiment, the loops encoded by the nucleic acid all have the sequence 5′-ANCA-3′.

In an embodiment, the nucleic acid comprises SEQ ID NO:2 or 3. In an embodiment, the nucleic acid comprises SEQ ID NO:2. In an embodiment, the nucleic acid comprises SEQ ID NO:3.

In an embodiment, the nucleic acid encodes from twelve to twenty four loops. In an embodiment, the nucleic acid encodes twelve loops. In an embodiment, the nucleic acid encodes twenty four loops.

Also provided is a nucleic acid encoding, in 5′ to 3′ order or 3′ to 5′ order:
(i) a CYC1 promoter;
(ii) a MS2 bacteriophage coat protein homo-dimer (MCP);
(iii) a first fluorescent protein;
(iv) a second fluorescent protein;
(v) a nuclear localization sequence (NLS);
(vi) a CYC1 terminator sequence.

Also provided is a nucleic acid encoding, in 5′ to 3′ order or 3′ to 5′ order:
(i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence.

In an embodiment, the nucleic acid comprises SEQ ID NO:4 or 32. In an embodiment, the nucleic acid comprises SEQ ID NO:4. In an embodiment, the nucleic acid comprises SEQ ID NO:32.

In an embodiment, the nucleic acid encodes (i) through (iv) in 5′ to 3′ order.

In an embodiment, the first fluorescent protein and second fluorescent protein have the same amino acid sequence.

In an embodiment, the first fluorescent protein and second fluorescent protein are a GFP or a tdTomato.

In an embodiment, the first fluorescent protein and second fluorescent protein are eGFP.

A kit is provided comprising two plasmids, (1) and (2), wherein:
(1) encodes from seven to twenty four loops of 5′-ANC/UA-3′, each wherein the 5′ end of each loop is connected to a sequence of eight nucleotides, seven of which nucleotides complementary to a sequence of seven nucleotides connected to the 3′ end of the same loop, such that a stem and loop structure is formed for each loop, and wherein each loop is separated from each adjacent loop by a nucleotide sequence of 40-55 nucleotides, and
(2) encodes, in 5′ to 3′ order or 3′ to 5′ order:
(A) (i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence, or
(B) (i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence; and
(3) instructions for use in visualizing an RNA of interest in a eukaryotic cell.

Exemplary plasmid sequences are set forth in SEQ ID NOS:2, 3, 4 and 32. In embodiments, (1) is SEQ ID NO:2 or 3. In embodiments, (2) is SEQ ID NO:4 or 32.

A kit is provided comprising two plasmids, (i) and (ii), wherein:
(a) encodes from seven to twelve loops of 5′-ANC/UA-3′, each wherein the 5′ end of each loop is connected to a sequence of eight nucleotides, seven of which nucleotides complementary to a sequence of seven nucleotides connected to the 3′ end of the same loop, such that a stem and loop structure is formed for each loop, and wherein each loop is separated from each adjacent loop by a nucleotide sequence of 45-55 nucleotides, and (b) encodes, in 5′ to 3′ order or 3′ to 5′ order:
(i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence, and
(iii) instructions for use in visualizing an RNA of interest in a eukaryotic cell.

Exemplary sequences of (a) and (b) are set forth in SEQ ID NOS:2, 3, 4 and 32. In embodiments, (a) is SEQ ID NO:2 or 3. In embodiments, (b) is SEQ ID NO:4 or 32.

Also provided is a method of detecting or of monitoring an RNA of interest in a yeast cell, the method comprising:
(i) inserting in a cell the nucleic acid, encoding from twelve to twenty four loops of 5′-ANC/UA-3′, as described herein into the 3′-UTR of a gene encoding the RNA of interest by homologous recombination and using Cre-Lox recombination so as to remove a marker gene so as to tag the RNA of interest with the twelve to twenty four loops;
(ii) transfecting the cell with a nucleic acid, encoding an MCP, as described herein so as to permit expression of a fusion protein comprising the MCP, NLS and the first and second fluorescent proteins and second fluorescent proteins so as to detect or monitor the RNA of interest.

Also provided is a method of detecting or of monitoring an RNA of interest in a yeast cell, the method comprising:
(i) inserting in a cell the nucleic acid, encoding from seven to twelve four loops of 5′-ANC/UA-3′, as described herein into the 3′-UTR of a gene encoding the RNA of interest by homologous recombination and using Cre-Lox recombination so as to remove a marker gene so as to tag the RNA of interest with the seven to twelve four loops;
(ii) transfecting the cell with a nucleic acid, encoding an MCP, as described herein so as to permit expression of a fusion protein comprising the MCP, NLS and the first and second fluorescent proteins
(iii) detecting or monitoring the movement and/or location of a fluorescent signal of the first and second fluorescent proteins so as to detect or monitor the RNA of interest.

Also provided is a method of detecting or of monitoring an RNA of interest in a yeast cell, the method comprising:
(i) inserting in a cell the nucleic acid, encoding from twelve to twenty four loops of 5′-ANC/UA-3′, as described herein into the 3′-UTR of a gene encoding the RNA of interest by homologous recombination and using Cre-Lox recombination so as to remove a marker gene so as to tag the RNA of interest with the twelve to twenty four loops;
(ii) transfecting the cell with a nucleic acid, encoding an MCP, as described herein so as to permit expression of a fusion protein comprising the MCP, NLS and the first and second fluorescent proteins
(iii) detecting or monitoring the movement and/or location of a fluorescent signal of the first and second fluorescent proteins so as to detect or monitor the RNA of interest.

Also provided is a method of detecting or of monitoring an RNA of interest in a yeast cell, the method comprising:
(i) inserting in a cell the nucleic acid, encoding from seven to twelve loops of 5′-ANC/UA-3′, as described herein into the 3′-UTR of a gene encoding the RNA of interest by homologous recombination and using Cre-Lox recombination so as to remove a marker gene so as to tag the RNA of interest with the seven to twelve loops;
(ii) transfecting the cell with a nucleic acid, encoding an MCP, as described herein so as to permit expression of a fusion protein comprising the MCP, NLS and the first and second fluorescent proteins and second fluorescent proteins so as to detect or monitor the RNA of interest.

Also provided is a method of detecting or of monitoring an RNA of interest in a eukaryotic cell, the method comprising:
(i) transfecting the eukaryotic cell with a plasmid or RNA that has been in vitro modified to encode from twelve to twenty four loops of 5′-ANC/UA-3′, as described herein, into the 3′-UTR of a gene encoding the RNA of interest
(ii) transfecting the cell with a nucleic acid encoding a MS2 bacteriophage coat protein homo-dimer (MCP) and a fluorescent protein;
(iii) detecting or monitoring the movement and/or location of a fluorescent signal of the first and second fluorescent proteins so as to detect or monitor the RNA of interest.

Also provided is a method of detecting or of monitoring an RNA of interest in a eukaryotic cell, the method comprising:
(i) transfecting the eukaryotic cell with a plasmid or RNA that has been in vitro modified to encode from seven to twelve loops of 5′-ANC/UA-3′, as described herein, into the 3′-UTR of a gene encoding the RNA of interest
(ii) transfecting the cell with a nucleic acid encoding a MS2 bacteriophage coat protein homo-dimer (MCP) and a fluorescent protein;
(iii) detecting or monitoring the movement and/or location of a fluorescent signal of the first and second fluorescent proteins so as to detect or monitor the RNA of interest.

In an embodiment, the nucleic acid encoding a MS2 bacteriophage coat protein homo-dimer (MCP) and a fluorescent protein is that described in (Wu, B et al, 2012).

In an embodiment, the eukaryotic cell is a mammalian cell. In an embodiment, the cell is a yeast cell. In an embodiment, in (i) the nucleic acid is inserted in the cell by way of a plasmid. In an embodiment, in (ii) the nucleic acid is transfected into the cell by way of a plasmid. In an embodiment, the fluorescent signal is monitored or detected by way of epifluorescence microscopy.

In an embodiment, in yeast cells (i) the nucleic acid is inserted in the cell by way of a PCR product or digested plasmid. In an embodiment, in (ii) the nucleic acid is transfected into the cell by way of a plasmid. In an embodiment, (iii) the fluorescent signal is monitored or detected by way of epifluorescence microscopy. In an embodiment, in mammalian cells (i) the nucleic acid is transfected in the cell by plasmid or RNA. In an embodiment, in (ii) the nucleic acid is transfected into the cell by way of a plasmid. In an embodiment, (iii) the fluorescent signal is monitored or detected by way of epifluorescence microscopy.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

To address this problem of spurious MBS-MCP system signals, the inventors designed a new MBS system that mimics the actual regulation of the endogenous untagged mRNA in eukaryotic cells by inhibition elicited by previous MBS versions, and then by engineering a novel MBS-MCP system that overcame the limitations. To minimize variability and preserve the original regulatory sequences, all MBS versions were tested by tagging the mRNAs at the endogenous loci. The system disclosed herein differs from previous systems by: (i) the controlled low expression of the MCP fused to a fluorescent protein, (ii) the reduced affinity of MBSV6 for MCP, (iii) the increased distance between MS2 loops, and (iv) reduction of the number of loops from 24 to 12 to facilitate degradation. Because MBSV6 decays with the same kinetics of the tagged mRNA, these modifications made the new MBS-MCP system an accurate reporter to image mRNAs from transcription to degradation. Finally, the inventors challenged this new MBS by following single mRNA decay for the highly unstable mRNAs, GAL1 and ASH1, and obtained successful results.

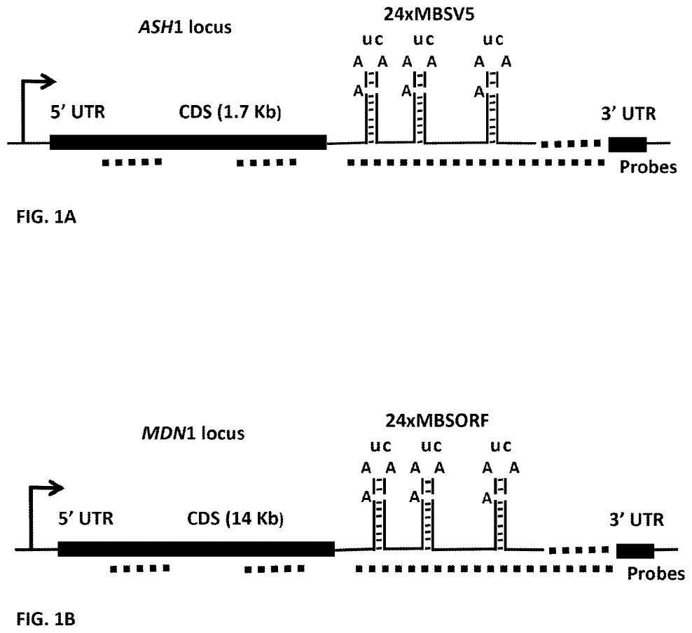

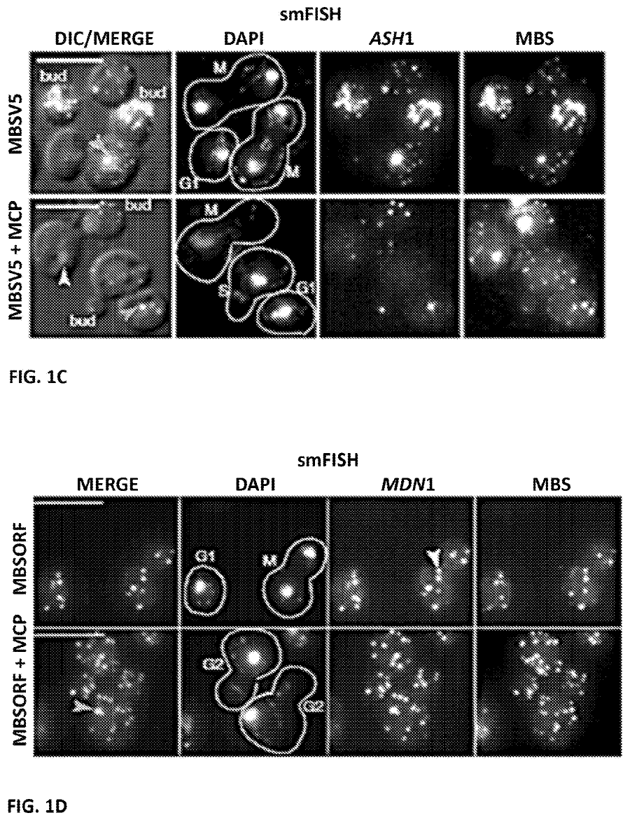

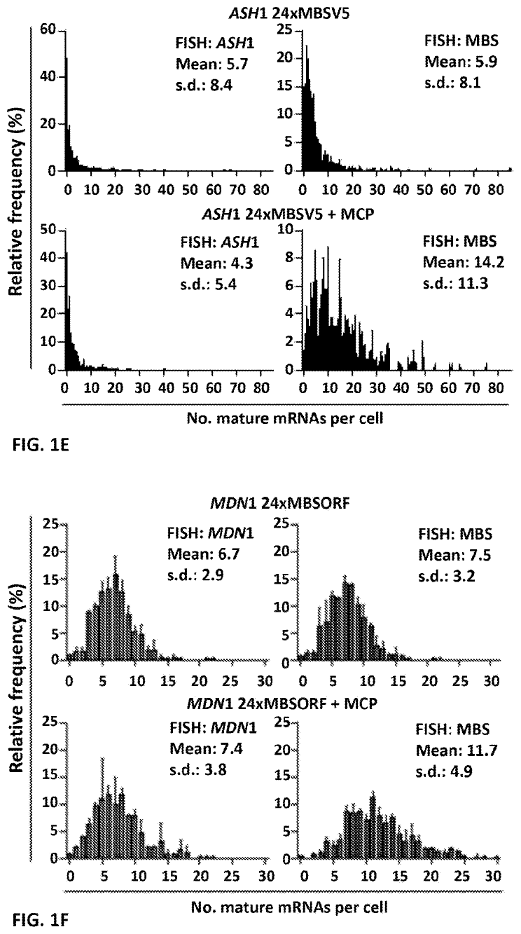

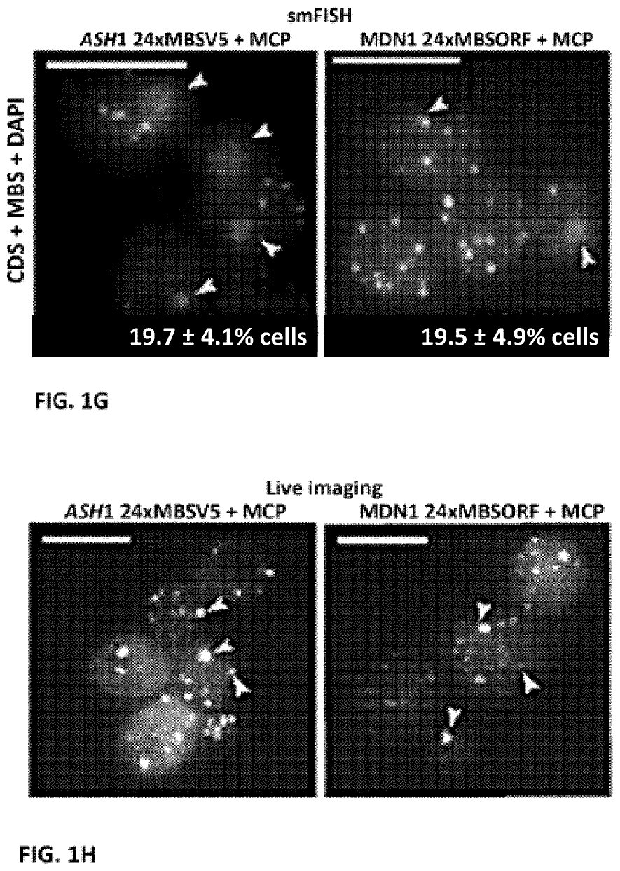

Characterization of the expression of mRNAs tagged with the MBS-MCP system: Previous studies indicating that the MBS-MCP system affected the degradation of tagged mRNAs used semi-quantitative ensemble approaches to assess this issue (eg. northern blot). To extend this analysis, we used two color smFISH to measure whether MBS-MCP tagged mRNAs accumulate 3′ decay fragments in S. cerevisiae. Two-color smFISH has several advantages over the northern blot approach i) RNA degradation is minimized because cells are intact ii) the integrity of the full length mRNA can be assessed at the single molecule level iii) the mRNA localization can be resolved and iv) cell-to-cell variation can be quantified. Two well characterized genes were analyzed: ASH1, a cell cycle regulated mRNA with a rapid turnover that localizes to the bud tip (Bertrand et al., 1998; Long et al., 1997b), and MDN1, a constitutively expressed mRNA (Hocine et al., 2013; Zenklusen et al., 2008). Both genes were endogenously tagged with 24×MBS in the 3′UTR. The most recent MBS version, MBSVS, contains non-repetitive stem loops (to avoid recombination) was used for ASH1 (Wu et al., 2015); FIG. 1A) and a previous version MBSORF that contains repetitive stem loops without stop codons (to avoid NMD) was used for MDN1 ((Hocine et al., 2013); FIG. 1B). Cells expressed the MCP fused to two GFP molecules from the constitutive Cytochrome C1 promoter (CYC1p) (Mumberg et al., 1995), which guaranteed an homogenous expression among cells. The addition of a nuclear localization signal to the MCP reduced the cytoplasmic background during live imaging (CYC1p-MCP-NLS-2×GFP, hereafter MCP).

The mRNA integrity was probed by two color smFISH simultaneously recognizing the coding sequence, CDS, (ASH1 or MDN1) or the MBS sequence (MBSVS or MBSORF) of the same mRNA (FIG. 1A-B). In an asynchronous population of cells, ASH1 mRNAs endogenously tagged with 24×MBSV5 properly localized to the bud tip during mitosis (FIG. 1C, top panels). Quantification using CDS probes showed similar expression level whether tagged or not, with or without MCP. However, in cells expressing MCP, two color smFISH showed an increased number of MBS molecules, without corresponding signal for ASH1 CDS, demonstrating an accumulation of 3′ decay fragments (FIGS. 1C and 1E). The number of MBS fragments varied among cells and was, on average, 2.3 times higher than the full length ASH1 mRNA (FIG. 1E). We obtained similar results for MDN1 mRNAs tagged with 24×MBSORF. The expression of endogenous MDN1 is constitutive with a Gaussian distribution that ranges from 0 to ˜15 mRNAs per cell (Zenklusen et al., 2008). However, the number of MBS doubled in the presence of the MCP relative to the mRNAs quantified with the CDS probes (FIGS. 1D and 1F).

In about 20% of both ASH1 and MDN1 tagged yeast strains expressing MCP, bright aggregates were seen containing only the MBS sequence that did not hybridize to the CDS probes (FIG. 1G). These MBS aggregates resulted from the accumulation of single MBS sequences protected from degradation by MCP. Strains not expressing MCP showed a strong correlation between the number of single mRNAs quantified with the CDS probes or MBS probes (Pearson coefficient for ASH1 r=0.91, MDN1 r=0.76) that was reduced in strains expressing MCP (ASH1 r=0.56, MDN1 r=0.23). This resistance to degradation was underestimated because cells with MBS aggregates were not included in the analysis since the fluorescent signal was saturated impeding the quantification. Moreover, reducing the number of MBSVS to twelve did not improve the degradation defect as observed by ASH1 and MBS smFISH.

Live imaging was consistent with the smFISH results. Strains expressing MCP with tagged ASH1 or MDN1 revealed that 20% of the cells contained MBS aggregates that were less mobile and brighter than single molecules (FIG. 1H). These aggregates were not only MCP because they required an MBS-tagged mRNA. ASH1 MBS fragments were found in all cells, regardless of the cell cycle phase, demonstrating that degradation was dysregulated. Hence, the use of the available MBS-MCP systems in living yeast can lead to false conclusions about mRNA expression and localization and mRNA tagging should be validated using two color smFISH experiments. These observations suggested that the tight binding between the MCP and MBS may block the access of the cytoplasmic exonuclease Xml, to the RNA (Garcia and Parker, 2015, 2016; Haimovich et al., 2016). Consequently, the kinetics of MBS fragment degradation would be slower than that of the CDS, leading to the formation of single MBS fragments that accumulate as aggregates. Consistent with the hypothesis that MBS aggregates form because of an mRNA degradation defect, only full-length ASH1 mRNAs but not MBS fragments localized to the bud tip, suggesting that the binding of the MCP to the MBS allows proper expression and localization of ASH1 mRNA but delays the degradation of the MBS sequence. The severity of the phenotype is aggravated for highly regulated and short-lived mRNAs, like ASH1, compared to constitutively expressed genes like MDN1. To overcome this problem, we designed degradable MBS versions.

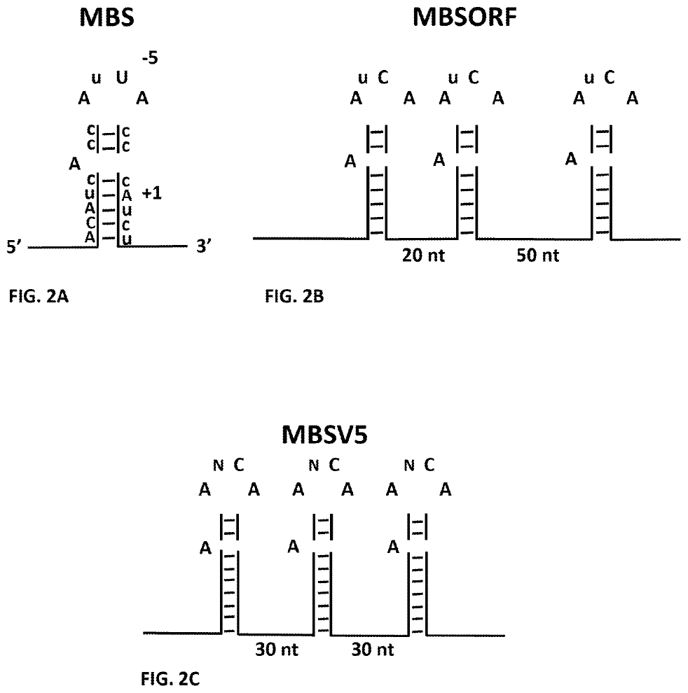

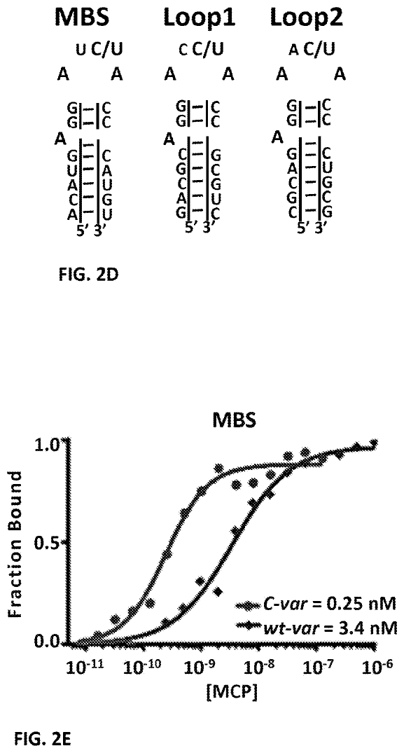

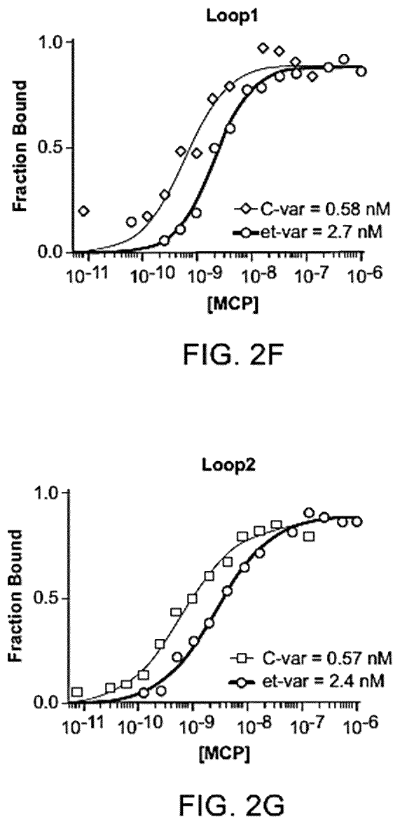

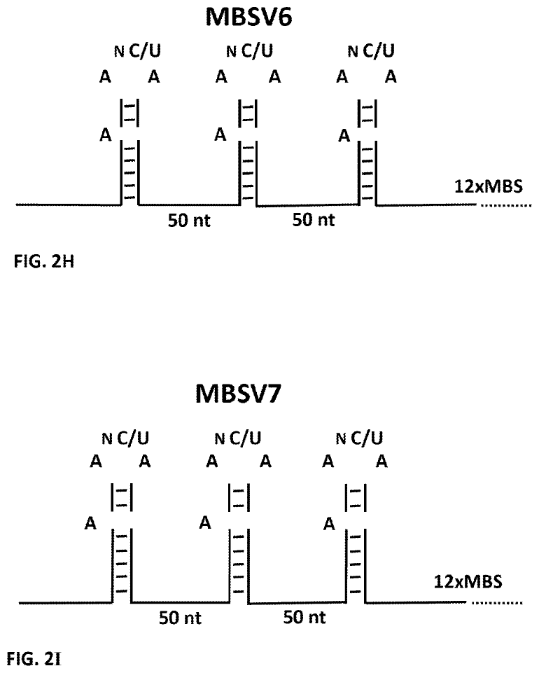

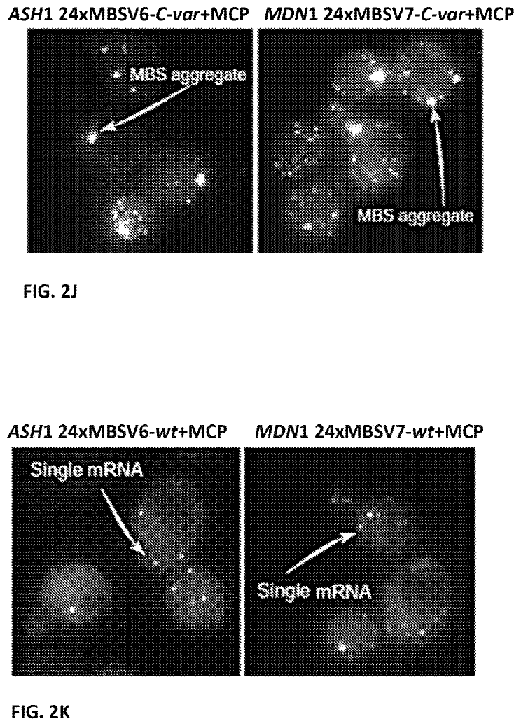

Design of a new MBS-MCP system faithfully recapitulating mRNA kinetics—We considered four variables that could influence the ability of the MS2 array to block degradation by Xrn1 when MCP is bound. First, the loop sequence can affect the affinity of MCP for the stem. Previous in vitro characterization of the MBS-MCP interaction identified the MS2 C-variant, in which the wild-type (wt) uridine at position −5 of the loop is substituted by a cytosine (FIG. 2A), increasing the affinity of the MCP tenfold (Kd from 10 nM to 1 nM) and decreasing the dissociation kinetics about 90 times (Lowary and Uhlenbeck, 1987; Valegard et al., 1994; Valegard et al., 1997). The current MBSVS and MBSORF versions have a U to C change in position −5 of all loops (FIG. 2A-C). We compared the high affinity C-variant loops to the wt-loops (FIG. 2D). The affinities of the MBS-MCP binding were analyzed by electrophoretic mobility shift assay (EMSA). As a control, we used the original loop sequence from the bacteriophage with a U or a C at position −5. In addition, C or U variants were generated for two MS2 loops (Loop 1 and Loop2), each of them with a different stem sequence (FIG. 2D). Apparent equilibrium dissociation constants (Kd) were extracted from plots of MCP bound versus free (FIG. 2E-G).

For the three loops tested, MBS, Loop1 and Loop2, the Kd values of wt-variants were in the nanomolar range while the C-variants were in the sub-nanomolar range (FIG. 2, E-G). Therefore, the C to U mutation was sufficient to reduce the affinity of the MCP for the MBS independent of the sequence.

We made three additional alterations we anticipated would allow increased degradation of the RNA by Xml. First, the stem of MBSVS was reduced to 7 nt to further decrease affinity. Second, we increased the length of the linkers from 30 nt to either 40 or 50. Finally, we reduced the stem-loops to 12 compared to 24 to provide less substrate for degradation.

Based on these variables, we generated eight constructs: (1) 12×MBSV7 wt-variant with 12 different loops, but with a U at position −5, interspaced by 40 nt linkers; (2) 12×MBSV7 C-variant, with the same sequence but with C instead of U at position −5 in the 12 loops; (3) 12×MBSV6 wt-variant, the same loop sequences of (1) but with 50 nt linkers; (4) 12×MBSV6 C-variant, with the same sequence as (3) but with C instead of U at position −5 in the 12 loops. For comparison, a 24×MBS variant was generated by duplication of the 12 loop cassette (FIGS. 2H-I). Additionally, to avoid NMD, STOP codons were avoided in all frames. Cells co-expressing MCP with ASH1 24×MBSV6 or MDN1 24×MBSV7 Cvariant in the 3′ UTR, had MBS aggregates in the cytoplasm (FIG. 2J). These observations indicated that the high affinity binding of MCP to C-variant MBS triggers the formation of MBS aggregates, independent of the stem loop sequences and the length of the stem or the linkers. In contrast, no MBS aggregates were observed in cells co-expressing the ASH1 24×MBSV6 or MDN1 24×MBSV7 lower affinity wt-variants (FIG. 2K). Most importantly, single mRNA molecules could be detected in vivo. Therefore, we proceeded with the molecular characterization of the wt-variants, MBSV6 or MBSV7.

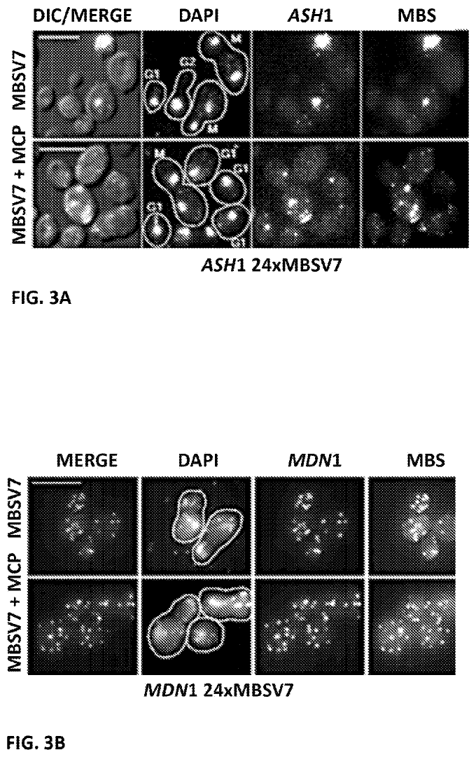

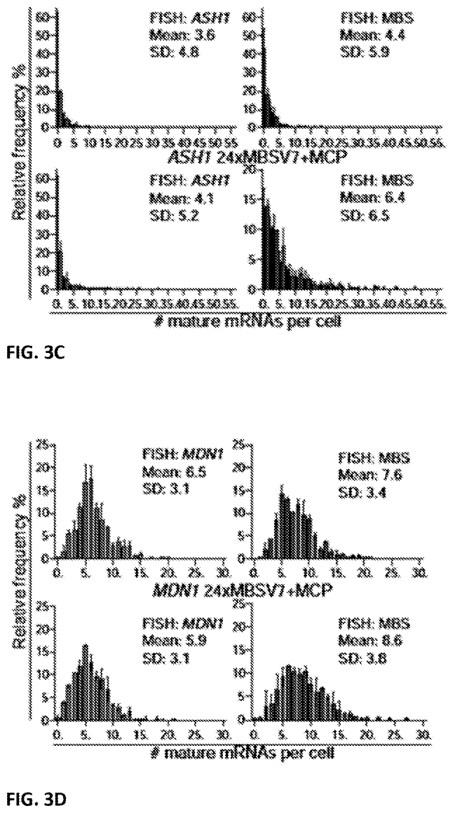

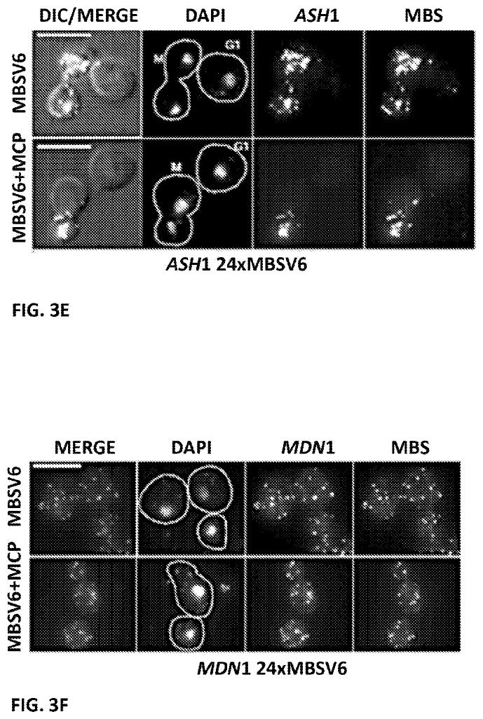

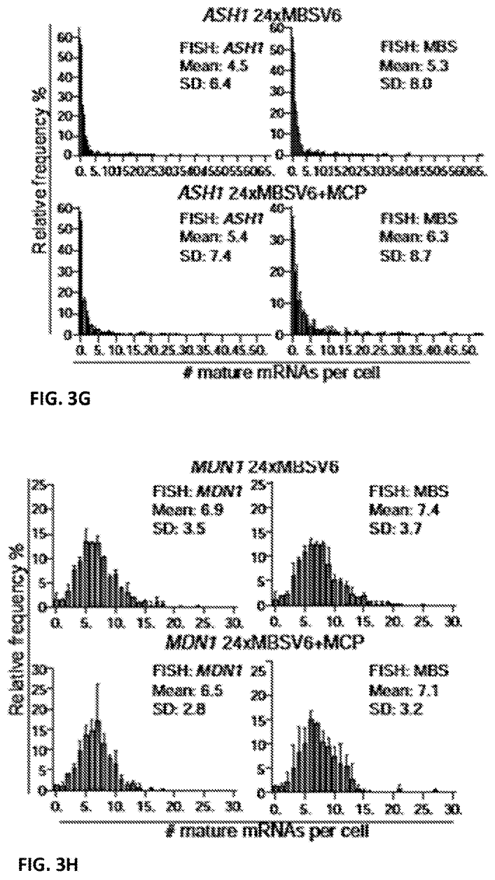

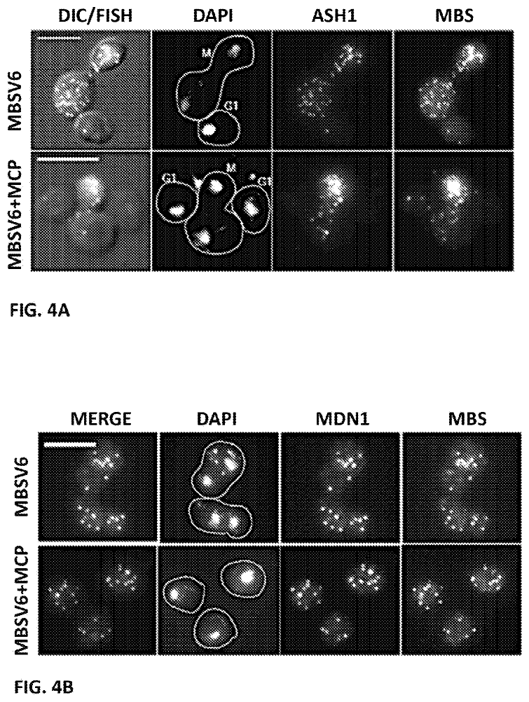

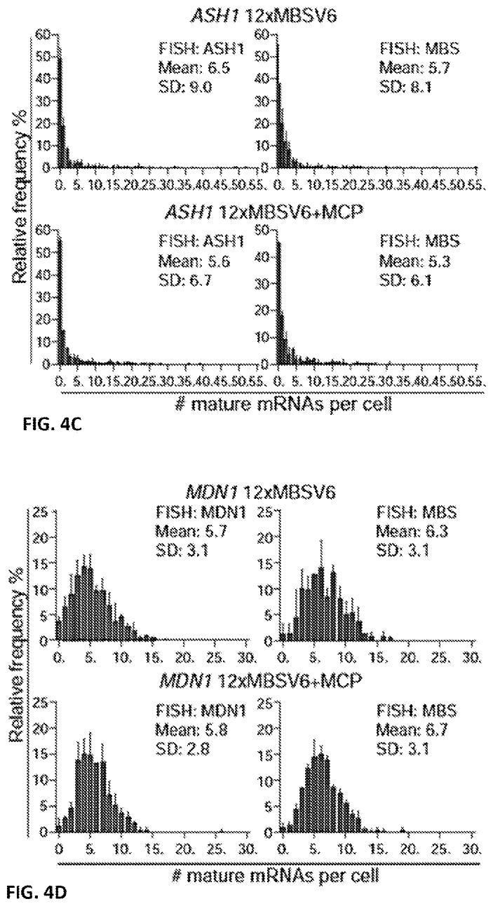

smFISH of endogenous mRNAs tagged with wt-variants MBSV6 and MBSV7—The key experiment to see how these new MBS-MCP systems affected degradation by Xrn1 was to determine if tagged mRNAs were full length. Two color smFISH was used to analyze yeast strains tagged in the 3′UTR with 24×MBSV6 or 24×MBSV7. The insertion of 24×MBSV7 in MDN1 or ASH1 mRNAs did not affect their expression (FIG. 3A-D). Co-expressing MCP reduced the percentage of cells forming small MBS aggregates to 2%, compared to 20% observed for previous MBS versions (data not shown). Reducing the insertion further to 12 stem loops (12×MBSV7) still did not prevent the accumulation of MBS aggregates in the presence of MCP (data not shown). Therefore, the MBSV7 wt, with lower affinity and 40 nucleotide linkers, ameliorated, but did not eliminate the number of cells containing MBS fragments and aggregates. In contrast to MBSV7, tagging ASH1 and MDN1 with 24×MBSV6, did not lead to the formation of MBS aggregates when co-expressing MCP and almost no fragments. Moreover, the distributions and the mean of the CDS and the MBS single molecules were similar (FIG. 3E-H). The Pearson correlation analysis of the CDS with MBSV6 or MBSV7 molecules in single cells confirmed this improvement. Reducing the stem loops from 24 to 12 for MBSV6 yielded similar results (FIG. 4A-D). Quantification of ASH1 and MDN1 mRNAs by two-color smFISH was essentially the same between the numbers of MBS and CDS molecules in cells expressing MCP, with no extra fragments (non-parametric T test, Mann Whitney test) (FIGS. 4C and 4D). To confirm that mRNAs tagged with the new MSBV6-MCP system were full length, northern blot analysis was performed using a probe hybridizing to the ASH1 mRNA after the site of MBS integration. We compared the endogenous ASH1 mRNA with tags MBSVS, MBSV7 and MBSV6, either with 24 or 12 stem loops. Consistent with previous observations, ASH1 mRNAs tagged with C-variant 24×MBSV5 and co-expressed with MCP showed the accumulation of MBS fragments (Garcia and Parker, 2015, 2016; Haimovich et al., 2016). In contrast, ASH1 mRNAs tagged with wt-variants MBSV7 and MBSV6, either 24 or 12 MBS showed a significant reduction in the accumulation of MBS fragments, as observed by smFISH.

Therefore, tagging of cycling ASH1, or constitutive MDN1 genes with the MBSV6 system recapitulated the endogenous pattern of expression and the expected cellular localization of the full-length mRNA, even when bound by MCP.

Characterization by live imaging of mRNAs tagged with MBSV6 in yeast and U2OS cells To compare the brightness of single mRNAs in live cells between the old and new constructs, we used mixed cultures from strains expressing either MDN1 24×MBSORF or MDN1 24×MBSV6. Cells were differentiated by the nuclear pore protein Nup49 tagged with tdTomato in the strain expressing MBSV6. The average intensity of single MDN1 mRNAs was determined for each strain and no significant (P=0.6753) differences were found. The bright cytoplasmic MBS aggregates in the 24×MBSORF-MCP expressing cells were excluded from the analysis because they were so bright, they were visible in the NUP (red) channels. The number of mRNAs per cell for both strains was consistent with the quantifications obtained by smFISH, indicating that the decreased affinity of MCP for the wt-variant did not compromise the detection of single mRNA molecules by live imaging.

MBSV6 could also be used to visualize single mRNAs in mammalian cells as with the previously characterized MBSVS system (Wu et al., 2016; Wu et al., 2015). A reporter gene (coding for BFP) tagged with 24×MBSV5 or 24×MBSV6 co-expressed with tdMCP in U2OS cells after transient transfection, (Wu et al., 2016)) showed transcription sites in the nucleus (yellow circles) and single mRNAs in the cytoplasm (orange circles). The intensities of the mRNAs tagged with the two systems were similar and had a Gaussian distribution, as expected for single mRNAs molecules. These results suggested that although in mammalian cells MBS aggregates were not observed for the reporter, the MBSV6-MCP technology could be a further improvement to analyze highly unstable mRNAs.

mRNA localization during stress induced by glucose starvation—The MBS-MCP system has been used to co-localize mRNAs with Processing Bodies (PBs), stress granules or peroxisomes during stress conditions (Haim-Vilmovsky et al., 2011; Haim-Vilmovsky and Gerst, 2009; Haim et al., 2007; Sheth and Parker, 2003; Simpson et al., 2014; Zid and O'Shea, 2014). However, recent experiments suggested that only the MBS fragments, but not the CDS of the tagged mRNA, colocalize with PBs (Heinrich et al., 2017). To ensure that during stress the MBSV6 system eliminates the possibility of misinterpretation in mRNA localization experiments, we used live imaging to visualize ASH1 or MDN1 mRNAs and PBs markers during glucose starvation. We monitored PB formation during glucose starvation by co-expressing the de-capping co-factor Edc3 fused to mCherry (Haimovich et al., 2013; Kshirsagar and Parker, 2004) in cells where ASH1 or MDN1 were tagged with either the previous 24×MBSMCP system (MBSVS or MBSORF) or 24×MBSV6-MCP. Cells expressing ASH1 or MDN1 tagged with the previous system showed cytoplasmic MBS aggregates even before glucose deprivation in both channels and substantial bleed-through that could affect interpretation of co-localization with a second labeled component. Edc3-mCherry granules started to form but no co-movement with MCP aggregates was observed after 10 minutes of glucose starvation. However, repeated interactions occurred over time between Edc3-mCherry and MCP aggregates. These results suggested that the PBs may recognize MCP aggregates as potential targets even if enclosed mRNAs were MBS fragments. However, glucose starvation did not induce the formation of MCP aggregates in cells expressing ASH1 and MDN1 mRNAs tagged with 24×MBSV6-MCP, and did not recruit single ASH1 and MDN1 mRNA molecules to PBs. Two-color smFISH confirmed that ASH1 and MDN1 mRNAs tagged with 24×MBSV6-MCP were full length throughout glucose starvation. The stress response was confirmed by smFISH for the heat shock mRNA HSP104 (Zid and O'Shea, 2014). These results validate the use of the MBSV6-MCP system for analyzing mRNA regulation during stress conditions.

Following rapid changes in mRNA degradation—Changing the carbon source produces drastic adjustments in the transcriptome of yeast cells (Lohr et al., 1995). One of the most sensitive genes to these changes is GAL1, which encodes the galactokinase involved in the first step of galactose metabolism. Shifting cells from glucose to raffinose creates a preinduced state, leading to a rapid induction of the GAL1 mRNA upon galactose addition (Hsu et al., 2012).

Conversely, washing out the galactose and adding glucose inhibits GAL1 transcription and induces GAL1 mRNA decay (Hsu et al., 2012), allowing determination of GAL1 mRNA half-life (t1/2). Two yeast strains expressing the GAL1 mRNA tagged with 24×MBSV6 with or without MCP were analyzed by two color smFISH. As expected, cells growing in raffinose do not express GAL1 mRNA.

Addition of 0.2% galactose for 30 minutes triggered similar induction of GAL1 mRNA expression quantified with either CDS or MBS probes (FIG. 6B and S6A). After switching back from galactose to glucose, the number of CDS and MBS single molecules declined similarly over time in presence or absence of MCP, reaching undetectable levels after sixty minutes. The high levels of mRNA produced led to small MBS aggregates in a minor population analyzed separately.

To calculate the t_{1/2} of GAL1 mRNA in cells without aggregates, the average number of single mRNA molecules per cell, quantified with both CDS and MBS probes at each time point after glucose addition, was normalized to that before glucose. A wt GAL1 yeast strain grown in the same conditions was used as control. The data collected for each strain were fitted to a single exponential decay model to calculate t_{1/2}. The endogenous GAL1 had a t_{1/2}=14 min Tagging of GAL1 with 24×MBSV6 shortened its half life to t_{1/2}=11 minutes. Notably, the curves obtained for the CDS and the MBS probes were practically identical. In presence of MCP, the t_{1/2} obtained with the MBS probes was t_{1/2}=17 min, on average 1.3 times longer than the one obtained with the CDS probes (t_{1/2}=14 min) or the endogenous GALL In cells co-expressing GAL1 12×MBSV6 and MCP, the CDS and the MBS sequence were degraded simultaneously, in contrast to 12×MBSV5. The real time decay of GAL1 12×MBSV6—MCP induced by glucose was observed by live imaging during an hour. Because cells in galactose contained mRNAs at the same time points as the glucose repression, it was concluded that the reason for the mRNA signal disappearance was degradation instead of photo-bleaching.

Remarkably, no MBS aggregates were observed in the 12×MBSV6 strain while in 12×MBSV5, the percentage of cells with MBS aggregates increased over time from 15%, at 15 minutes of recovery, to 30% at 60 minutes. Instead, the amount of single MBS molecules per aggregate as well as the percentage of cells with aggregates decreased over time for the GAL1 tagged with 24×MBSV6 strain. Cells with MBS aggregates had more MBS single molecules than CDS molecules at any time point during recovery (data not shown). It is possible that MBSV6 aggregates formed in either cells with slower mRNA degradation or cells with stronger GAL1 induction. Therefore, by reducing the number of MBSV6 repeats from 24 to 12 we could measure precisely the abundance and degradation of highly induced and regulated genes in cells undergoing rapid metabolic adjustments.

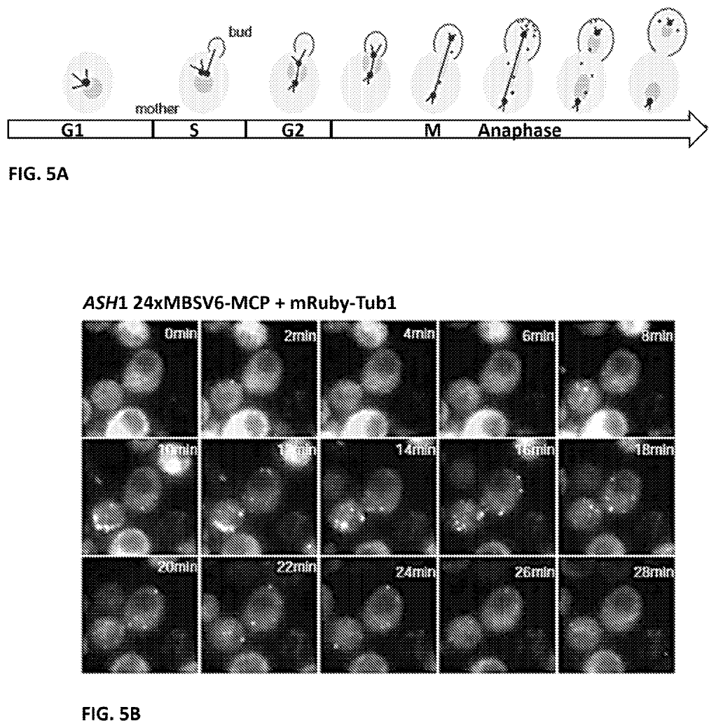

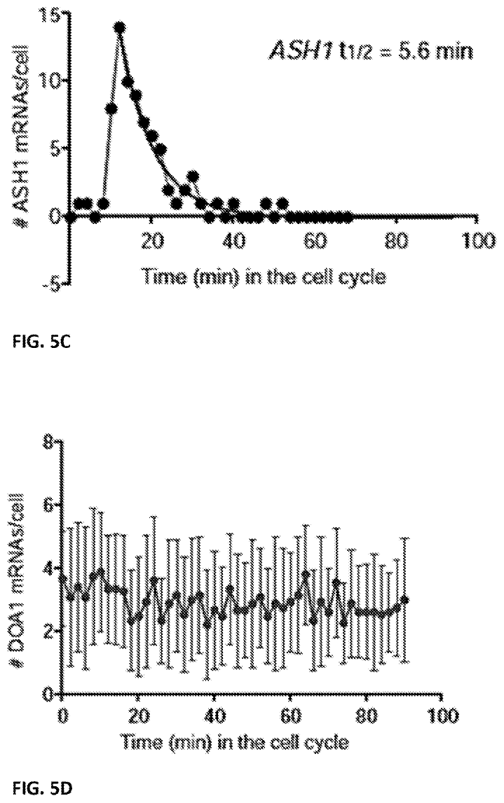

ASH1 mRNA cell cycle expression—Cell cycle regulated genes require precise regulation of their synthesis and degradation (Trcek et al., 2011). In yeast, ASH1 mRNA expression is temporally restricted to anaphase and the mRNA localizes to the bud tip where it is locally translated to control mating type switching (Heym and Niessing, 2012; Long et al., 1997a; Long et al., 1997b). To quantify ASH1 mRNAs during the cell cycle, ASH1 was tagged with 24×MBSV6 and cell cycle progression was monitored with the TUB1 gene tagged with mRuby (FIG. 7A). The microtubules extend from the spindle pole body (SPB) between the mother and daughter cell, participating in chromosome separation and mitosis (Pereira and Schiebel, 2001); FIG. 5A). ASH1 mRNA expression was preceded by microtubule stretching, allowing the clear identification of mother-daughter pairs and cell cycle phase (FIG. 5A-5B). The number of single ASH1 mRNAs for each time point was counted (a representative cell is shown and quantified in FIGS. 5B and 5C). The quantification of 21 cells showed that ASH1 mRNA was expressed during 30 minutes corresponding to anaphase of mitosis. We measured t1/2 (5.6-6.1 min), that was consistent with recent reports that the full cycle of ASH1 mRNA lasted about 30 min and the mean half-life was 6.3±1 min at 30° C. (FIG. 5C) (Eser et al., 2014). We observed bursts of transcription in the mother that were followed by localization of the mRNAs to the bud tip; surprisingly before the end of mitosis a second burst of accumulation occurred in the bud tip, presumably from transcription in the daughter. The ASH1 mRNAs in the daughter cell stayed for a prolonged period before degradation.

To investigate the origin of the second accumulation, we analyzed ASH1 transcription during the cell cycle. To facilitate the visualization of the transcription site, a kanamycin resistance gene was added to the ASH1 24×MBSV6 3′UTR, in order to increase the residence time of the nascent transcripts. To identify the nucleus, and the transcription site therein, we co-expressed 2xmCherry fused to an NLS. Simultaneous imaging of MCP and NLS-2xmCherry, revealed that transcription occurred both in the mother and the daughter cell nuclei. This suggests that mRNA transport from the nucleus to the bud tip may be regulated differently between mRNAs produced in the mother or the daughter cell.

To demonstrate that the disappearance of the mRNA signal is due to mRNA decay and not to photo-bleaching, we used the live imaging method to follow an mRNA with a longer half-life. The DOA1 gene that is constantly expressed throughout the cell cycle (Trcek et al., 2011) was tagged with 24×MBSV6. The number of single DOA1 molecules was constant over the cell cycle with an average of 2.9±1.1 mRNAs/cell (FIG. 5D). Accordingly, two-color smFISH experiments with DOA1 or MBSV6 probes reported 3.6±2.9 mRNAs/cells and a Gaussian distribution, characteristic of constitutive genes. Thus, most of the mRNAs expressed in the cell could be visualized in vivo by the MBSV6-MCP system. These experiments demonstrated that the MBSV6 system provided the temporal and spatial resolution required to quantify the expression of mRNAs from birth to death in cells grown in optimal or stress conditions.

### DISCUSSION

The MBS-MCP system has been extensively used to study gene expression regulation by following endogenously tagged mRNAs (Vera et al., 2016). The use of MBSVS with the C-variant is appropriate for mammalian cells where mRNAs with long half-lives are investigated. However, in cases where the mRNA half-life is short and degradation of the MBS becomes rate limiting, the MBS can accumulate (Garcia and Parker, 2015, 2016; Haimovich et al., 2016; Heinrich et al., 2017). Therefore, we engineered and characterized a new MBS-MCP system that faithfully recapitulates the life cycle of the mRNA while preserving single molecule resolution. The approach can be used to validate mRNAs tagged with orthologous systems, such as PP7 (Chao et al., 2008) or U1A in yeast (Caponigro et al., 1993). Other systems to detect endogenous mRNA molecules, like the Spinach aptamer (Guet et al., 2015) or the Cas9 System (Nelles et al., 2016), do not yet reach the single-molecule sensitivity obtained by the MBS-MCP system.

The key improvement made for the application of the MBS-MCP system to short-lived mRNAs was to reduce the binding affinity between the MBS and MCP. Strikingly, the modification of one nucleotide at position −5 of the MS2 loop, from the wt uridine to cytosine in the C-variant, was sufficient to reduce the Kd about 10 fold, regardless of the stem loops sequence (FIG. 2E-G) (Lowary and Uhlenbeck, 1987).

Importantly this modification did not affect the brightness and detection of single molecule mRNAs suggesting that the recycling of MCP molecules could compensate for the decrease in affinity, and might reduce photobleaching by replenishment of the MCP (FIG. 5C). This MBS-MCP system, MBSV6, was inserted into the 3′UTRs of ASH1, MDN1 and DOA1 mRNAs, at the endogenous loci to preserve the regulatory elements. In all cases, MBSV6 faithfully reported the lifetime of the full-length mRNA by reducing the accumulation of MBS fragments and preventing the formation of MBS aggregates. Therefore, for imaging or biochemical experiments the MBSV6 avoids the risk of experimental artifacts derived from the accumulation of intermediate degradation products. Nonetheless, for highly expressed genes like GAL1, 24×MBSV6 still triggered the formation of small MBS aggregates in 5% of the cells co-expressing MCP, indicating that degradation of highly expressed long stem loops (24×) is challenging for yeast cells.

Shorter stem loops (12×) however avoided the formation of degradation intermediates and enabled the degradation of the CDS and the MBS with the same kinetics (FIG. 6C). Further work will elucidate if the accumulation of RNA-protein aggregates is a mechanism to degrade structured mRNAs, such as the one bearing poly-G tracts, already known for blocking Xml activity (Sheth and Parker, 2003).

Previous reports suggested that the MBS system is particularly sensitive to stress conditions (Heinrich et al., 2017). Upon stress induction, coordinated recruitment of mRNAs tagged with MBSV6 to PBs was not observed. Because mRNAs in the cytoplasm are efficiently translated (Ingolia et al., 2009), long mRNAs like MDN1 are likely protected by ribosomes from recruitment to PBs. Conversely, the ASH1 mRNA translation is inhibited until the mRNA localizes to the bud tip, suggesting that other mechanisms could exist to protect these mRNAs from going to PBs. Further work is required to re-evaluate the role of PBs, and other cytoplasmic structures, in coordinating the cellular response to stress. For this purpose, the MBSV6-MCP offers the spatial and temporal resolution to elucidate the interactions of mRNAs and RNA binding proteins, forming cytoplasmic mRNP granules. These two color experiments would also provide insights on mRNA decay regulation and on the kinetics of assembly and disassembly of stress granules.

MBSV6 allows quantifying single mRNA in living cells during their entire life-cycle. Time-lapse imaging showed that the ASH1 mRNAs were produced from a transcriptional burst rapidly occurring during anaphase both in the mother and in the daughter nuclei. ASH1 mRNAs are then correctly localized to the bud tip, where they remained for as little as 8 minutes during mitosis (FIG. 7B), emphasizing the precise temporal resolution of this approach. The improved sensitivity of this system allowed us to observe a second burst of transcription in the daughter nucleus that led to mRNAs that could be observed in the cytoplasm for few minutes after mitosis, although mRNAs did not remain in the mother cell. These results suggest either that ASH1 mRNAs are efficiently transported from the mother to the daughter cell, or they are selectively degraded in the mother. Further experiments using the MBSV6 will elucidate factors required to regulate the coupling between ASH1 mRNA localization, translation and decay.

The value of this new MS2 system is to provide a new capability for imaging and measuring the regulatory events of the entire RNA lifetime without perturbation. In particular, the decay events of single RNAs in single cells can now be elucidated with temporal and spatial resolution sufficient to study the localization and function of highly unstable RNAs, such as non-coding or regulatory RNAs.

### Materials and Methods

Yeast strains construction—Yeast strains were constructed in the BY4741 or BMA64-1A background. All strains where a gene of interest was tagged with MBSs in the 3′UTR, right after the STOP codon, were prepared as follow: PCR amplification of the MBS insert (see plasmids in Table 2) followed by the kanamycin resistance gene, flanked by Loxp sequences, was performed with oligos (see oligos Table 3) containing homology sequences (50-70 nt) for the specific gene. For all strains, the Kanamycin resistance gene was removed by expressing the CRE recombinase under the control of the GAL1 promoter. Genomic DNA was extracted using standard techniques and PCR amplification of the 3′ UTR was loaded on a gel and sent for sequencing to verify the size of the insert.

Plasmids construction—The new MBS sequences, wt or C-variants, 12×MBSV6 and 12×MBSV7 were synthetized by Genscript. To obtain the 24×MBSV6 and 24×MBSV7 we cloned the 12×MBS V6/V7 in tandem by using restriction enzymes BamHI and BgIII. Orientation of the insert was confirmed by sequencing. The 12×MBS or 24×MBS variants were then transferred in the yeast vector containing the Kanamycin resistance gene

flanked by Loxp by using the restriction enzymes BamHI SaII. The plasmid pET296 was generated by inserting the CYC1p, amplified from genomic DNA of BY4741, with flanking restriction enzymes XhoI and BamHI. The NLS from SV40 was added at the C-terminus of the MCP coding sequence by PCR amplification using a reverse oligo containing the NLS sequence, flanked by restriction enzymes BamHI and AgeI. In the SINAPV5 plasmid the sequence of 24×MBSV5 was replaced by digesting with AgeI and ClaI restriction enzymes and inserting within the same site 24XMBSV6 amplified by PCR. For EMSAs a C-terminal His Tag was added by PCR and MCP-His was cloned using BamHI and HindIII sites into a pMalc derivative that contains a Tobacco Etch Virus (TEV) site after the maltose-binding protein (plasmid pET336-Table 1).

smFISH probes preparation—ASH1, DOA1, GAL1, MDN1, MBSVS, MBSV6, MBSV7 probes were designed using the Stellaris™ Probe Designer by LGC Biosearch Technologies and purchased from Biosearch Technologies. HSP104 and MBSORF probes were synthetized by Invitrogen-Thermo Fisher, and labelled in the lab using Cy3 dyes (Amersham) as previously described (Trcek et al., 2012).

smFISH and image acquisition and analysis—Single molecule FISH (smFISH), was essentially performed as described in (Trcek et al., 2012) with the following modifications. Yeast strains were grown overnight at 25° C. in selective medium with 2% glucose. In the morning cells were diluted to OD600 0.1 and allowed to grow until OD600 0.3-0.4. Yeast strains tagged in the GAL1 gene were grown for twenty-four hours in SC-Leu supplemented with 2% Raffinose. At OD=0.3, GAL1 expression was induced with 0.2% galactose for thirty minutes and decay was induced by adding 4% glucose, as described in FIG. 5A. Cells were fixed by adding paraformaldehyde (32% solution, EM grade; Electron Microscopy Science #15714) to a final concentration of 4% and gently shacked at room temperature for 45 minutes. Cells were then washed 3 times with buffer B (1.2M sorbitol and 100 mM potassium phosphate buffer pH=7.5) and resuspended in 500 μL of spheroplast buffer (buffer B containing 20 mM VRC (Ribonucleoside—vanadyl complex NEB #S1402S), and 25U of Lyticase enzyme (Sigma #L2524) per OD of cells for about 10 minutes at 30° C. Digested cells were washed once with buffer B and resuspended in 1 mL of buffer B. 150 μL of cells were seeded on 18 mm poly-lysine treated coverslips and incubated at 4° C. for 30 minutes. Coverslips were washed once with buffer B, gently covered with ice-cold 70% ethanol and stored at −20° C. For hybridization, coverslips were rehydrated by adding 2×SSC at room temperature twice for 5 minutes. Coverslips were pre-hybridized with a mix containing 10% formamide (ACROS organics #205821000)/2×SSC, at room temperature for 30 minutes. For each coverslip the probe mix (to obtain a final concentration in the hybridization mix of 125 nM) was added to 5 μL of 10 mg/μL E. coli tRNA/ssDNA (1:1) mix and dried with a speed-vac. The dried mix was resuspended in 25 μL of hybridization mix (10% formamide, 2×SSC, 1 mg/ml BSA, 10 mM VRC, 5 mM NaHPO4 pH 7.5) and heated at 95° C. for 3 minutes. Cells were then hybridized at 37° C. for 3 hours in the dark. Upon hybridization coverslips were washed twice with pre-hybridization mix for 30 minutes at 37° C., once with 0.1% Triton X-100 in 2×SSC for 10 minutes at room temperature, once with 1×SSC for 10 minutes at room temperature. Nuclei were stained with 0.5 μg/mL DAPI in 1×PBS for 2 minutes at room temperature, washed with 1×PBS for 10 minutes at room temperature. Coverslips were mounted on glass slides using ProLong Gold antifade (Thermo Fisher). Images were acquired using an Olympus BX61 wide field epi-fluorescence microscope with a 100X/1.35NA UPlanApo objective. Samples were visualized using an X-Cite 120 PC lamp (EXFO) and the ORCA-R2 Digital CCD camera (Hamamatsu). Metamorph software (Molecular Devices) was used for acquisition. Z-sections were acquired at 200 nm intervals over an optical range of 8.0 μm. Image pixel size: XY, 64.5 nm. FISH images were analyzed using FISHQUANT (Mueller et al., 2013). Briefly, after background subtraction, the FISH spots in the cytoplasm were fit to a three-dimensional (3D) Gaussian to determine the coordinates of the mRNAs. The intensity and width of the 3D Gaussian were thresholded to exclude nonspecific signal. The average intensity of all the mRNAs was used to determine the intensity of each transcription site.

Sample preparation for live yeast fluorescence imaging Yeast cells were grown at 25° C. in synthetic selective medium. Exponentially growing cells (O.D. 0.2-0.4) were plated on coated Delta-T dishes (Bioptech-04200417C). The dishes coating was done by incubating with Concanavalin A 1 mg/ml (Cayman chemical company) for 10 minutes at room temperature. Excess liquid was aspirated and dishes were dried at room temperature. To activate Concanavalin A, dishes were incubated for 10 minutes at room temperature with a 50 mM CaCl₂ 50 mM MnCl₂ solution. Excess was removed and dishes dried at room temperature. Finally, dishes were washed once with ultrapure water (Invitrogen), and let completely dry at room temperature. Cells attachment was performed by gravity for 20 minutes at room temperature, excess liquid removed and substitution with fresh media.

Glucose deprivation was performed by growing cells co-transformed with plasmids MCP-2×GFP and Edc3—mCherry in double selective medium with—2% glucose overnight at 25° C. Cells were diluted in the morning and grown until OD600 0.3-0.4. Cells were plated on Concanavalin A coated dishes. Images were acquired before glucose starvation and then, keeping the dishes on the microscope stage with appropriate temperature control, washes were performed 6 times with 1 ml of medium without glucose. Cells were then kept in medium lacking glucose at 25° C. taking z-stacks every minute for 40 minutes.

Live cells fluorescence imaging and image analysis—The two-color simultaneous imaging of mRNAs and the appropriate cellular marker was performed on a modified version of the home-built microscope described in (Wu et al., 2016). Briefly, the microscope was built around an IX71 stand (Olympus). For excitation, a 491 nm laser (Calypso™, Cobolt) and a 561 nm laser (Jive™, Cobolt) were combined and controlled by an acoustic-optic tunable filter (AOTF, AOTFnC-400.650-TN, AA Opto-electronic) before coupled into a single mode optical fiber (Qioptiq). The output of the fiber was collimated and delivered through the back port of the microscope and reflected into an Olympus 150× 1.45 N.A. Oil immersion objective lens with a dichroic mirror (zt405/488/561rpc, 2 mm substrate, Chroma). The tube lens (180 mm focal length) was removed from the microscope and placed outside of the right port. A triple band notch emission filter (zet405/488/561m) was used to filter the scattered laser light. A dichroic mirror (T560LPXR, 3 mm substrate, Chroma) was used to split the fluorescence onto two precisely aligned EMCCDs (Andor iXon3, Model DU897) mounted on alignment stages (x, y, z, θ- and φ-angle). Emission filters FF03-525/50-25 and FF01-607/70-25 (Semrock) were placed in front of green and red channel cameras respectively. The two cameras were triggered for exposure with a TTL pulse generated on a DAQ board (Measurement Computing). The microscope was equipped with a piezo stage (ASI) for fast z-stack and a Delta-T incubation system (Bioptech) for live cell imaging. The microscope (AOTF, DAQ, Stage and Cameras) was automated with the software Metamorph (Molecular Devices). For two-color live cell imaging, yeast cells were streamed at 50 ms, Z plane was streamed, and Z-stacks acquired every 0.5 μm. Single molecule analysis was done on the maximal projected images using AIRLOCALIZE (Lionnet et al., 2011).

Recombinant Protein Preparation—Transformation of pET336 and purification were performed as previously described (Chao et al., 2008). In brief, constructs were transformed into Rosetta2 cells (EMD Millipore) and protein induction was performed for 4 hours at 37° C. Cell pellets were lysed by sonication in 50 mM Tris pH 7.2, 1.5M NaCl, 1 mM EDTA, 1 mM DTT supplemented with one Complete EDTA-free protease inhibitor tablet (Roche). After centrifugation, the soluble protein was first purified by amylase affinity chromatography (New England Biolabs) and subsequently by TALON affinity chromatography (Takara Bioscience).

Electrophoretic Mobility Shift Assay (EMSA)—Single stem loop fragments with 5′ fluorescein modification (Dharmacon) were deprotected as per the manufacturer recommendation. Prior to the experiment, RNA stocks were heated to 70° C. for 5 minutes then snap cooled on ice. The sequences of the RNAs used for these experiments are listed in Table 1.

TABLE 1
RNA used for EMSA

| Name | Variant | Sequence |
|---|---|---|
| MS2 | wt | ACATGAGGATTACCCATGT |
| MS2 | C | ACATGAGGATCACCCATGT |
| V6/V7 SL_1 | wt | GACGCAGGACTACCGCGTC |
| V6/V7 SL_1 | C | GACGCAGGACCACCGCGTC |
| V6/V7 SL_9 | wt | CGCAGAGGAATACCCTGCG |
| V6/V7 SL_9 | C | CGCAGAGGAACACCCTGCG |

(Top to bottom, SEQ ID NOS:5-10, respectively).

Complexes were monitored and quantified by EMSA as previously described (Chao et al., 2008). In brief, 100 pM RNAs were incubated at room temperature for three hours with 2 fold dilutions of MCP in 10 mM Tris, 100 mM NaCl, 0.1 mM EDTA, 0.01 mg/mL tRNA, 50 μg/mL heparin and 0.01% IGEPAL CA630. Complexes were then run using 5% native PAGE in 0.5× TBE and visualized using the Typhoon 9400 variable mode laser scanner (GE Healthcare).

RNA preparation and Northern blots—Total mRNA was isolated from yeast cultures grown at 25° C. in synthetic selective medium as described in (Caponigro et al., 1993). Northern blots were performed by resolving 10 μg total RNA on 1.5% formaldehyde agarose gel, transferring by capillary action to a Nytran membrane, and, probing blots with [32P] end-labelled oligonucleotide complementary to the 3′ UTR of ASH1 (5′-ACAAGGAGAGAAATGTACAATTGTTTCGTGATAATGTCTCTTATTAGTTG-3′) (SEQ ID NO:11) as described in detailed in (Passos and Parker, 2008). Blots were stripped and reprobed for the 7S RNA using the following probe oRP100 (5′-GTCTAGCCGCGAGGAAGG-3′) (SEQ ID NO:12). Blots were visualized using a phosphoimager.

Mammalian cell cultures—Human U2OS osteoscarcoma cell line (American Type Culture Collection HTB-96) stable expressing tdMCPGFP (Wu et al., 2012) were grown at 37° C. and 5% CO2 in DMEM supplemented with 10% fetal bovine serum, 4.5 g/L glucose and 1% penicillin-streptomycin. Cells were transient transfected with SINAPV5 (Wu et al., 2016) or SINAPV6 (Table 1) with lipofectamine 3000 twenty-four hours before being subjected to live imaging experiments.

TABLE 2
Plasmids used in this study

| | | Yeast | | |
|---|---|---|---|---|
| Code | Name | Marker | Reference | Notes |
| pDZ415 | 24xMS2ORF | KAN | Hocine S, Raymond P, Zenklusen D, | Addgene 45162 |
| | | | Chao J A, Singer R H. Nat Methods. | |
| | | | 2013 February; 10(2): 119-21. | |
| pET157 | YcpLac111 | LEU2 | Rizzardi et al Genetics. 2012 | Addgene 53249 |
| | | | October; 192(2): 371-84. doi: | |
| | | | 10.1534/genetics.112.142349 | |
| pET184 | pSH47 GAl1p | URA3 | Güldener U, Heck S, Fiedler T, | from Euroscarf |
| | CRE | | Beinhauer J, Hegemann J H. A new | |
| | ricombinase | | efficient gene disruption cassette for | |
| | URA3 | | repeated use in budding yeast. Nucleic | |
| | | | Acids Research 1996; 24, 2519-2524 | |
| pET185 | pSH62 GAl1p | HIS3 | Güldener U, Heinisch J, Köhler G J, | from Euroscarf |
| | CRE | | Voss D, Hegemann J H. A second set of | |
| | ricombinase | | loxP marker cassettes for Cre-mediated | |
| | HIS3 | | multiple gene knockouts in budding | |
| | | | yeast. Nucleic Acids Research 2002; | |
| | | | 30, e23 | |
| pET194 | p415 KAN | KAN | Wu, B., V. Miskolci, H. Sato, E. | |
| | 24xMS2V5 | | Tutucci, C. A. Kenworthy, S. K. | |
| | | | Donnelly, Y. J. Yoon, D. Cox, R. H. | |
| | | | Singer and L. Hodgson (2015). | |
| | | | “Synonymous modification results in | |
| | | | high-fidelity gene expression of | |
| | | | repetitive protein and nucleotide | |
| | | | sequences.” Genes Dev 29(8): 876-886. | |
| pET334 | p415 KAN | KAN | This study | |
| | 12xMS2V5 | | | |
| pET246 | pUC57 | XXX | This study | |
| | 12xMS2V6 | | | |
| | wt loop 50 nt | | | |
| | linker | | | |
| pET247 | pUC57 | XXX | This study | |
| | 12xMS2V6 | | | |
| | C-var loop 50 | | | |
| | nt linker | | | |
| pET248 | pUC57 | XXX | This study | |
| | 12xMS2V7 | | | |
| | wt loop 41 nt | | | |
| | linker | | | |
| pET249 | pUC57 | XXX | This study | |
| | 12xMS2V7 | | | |
| | C-var loop 41 | | | |
| | nt linker | | | |
| pET251 | p415 | KAN | This study | |
| | 12xMS2V6 | | | |
| | 50 nt linker | | | |
| | WT Loxp | | | |
| | KANr Loxp | | | |
| pET252 | p415 | KAN | This study | |
| | 12xMS2V6 | | | |
| | 50 nt linker C- | | | |
| | var Loxp | | | |
| | KANr Loxp | | | |
| pET255 | p415 | KAN | This study | |
| | 12xMS2V7 | | | |
| | 41 nt linker | | | |
| | WT Loxp | | | |
| | KANr Loxp | | | |
| pET256 | p415 | KAN | This study | |
| | 12xMS2V7 | | | |
| | 41 nt linker | | | |
| | C-var Loxp | | | |
| | KANr Loxp | | | |
| pET259 | pUC57 | XXX | This study | |
| | inverted | | | |
| | 24xMS2V6 | | | |
| | wt 50 nt | | | |
| | linker | | | |
| pET261 | pUC57 | XXX | This study | |
| | 24xMS2V6 | | | |
| | C-var 50 nt | | | |
| | linker | | | |
| pET262 | pUC57 | XXX | This study | |
| | 24xMS2V7 | | | |
| | wt loop 41 nt | | | |
| | linker | | | |
| pET263 | pUC57 | XXX | This study | |
| | 24xMS2V7 | | | |
| | C-vart loop 41 | | | |
| | nt linker | | | |
| pET264 | p415 | KAN | This study | |
| | 24xMS2V6 | | | |
| | 50 nt linker | | | |
| | WT Loxp | | | |
| | KANr Loxp | | | |
| pET265 | p415 | KAN | This study | |
| | 24xMS2V6 | | | |
| | 50 nt linker C- | | | |
| | var Loxp | | | |
| | KANr Loxp | | | |
| pET268 | p415 | KAN | This study | |
| | 24xMS2V7 | | | |
| | 41 nt linker | | | |
| | WT Loxp | | | |
| | KANr Loxp | | | |
| pET269 | p415 | KAN | This study | |
| | 24xMS2V7 | | | |
| | 41 nt linker | | | |
| | C-var Loxp | | | |
| | KANr Loxp | | | |
| pET296 | YcpLac111 | LEU2 | This study | NLS form SV40 |
| | CYC1p | | | |
| | 1xMCP-NLS- | | | |
| | 2xyeGFP | | | |
| pET316 | pHIS3p | HIS3 | Markus S M, Omer S, Baranowski K, | Addgene 50657 |
| | mRuby2- | | Lee W L. Traffic. 2015 July; 16(7): 773- | |
| | Tub1 + 3′UTR | | 86. doi: 10.1111/tra.12276 | |
| | HIS3 | | | |
| pET317 | pCUP1- | TRP1 | Li D, Song J Z, Shan M H, Li S P, Liu | Addgene 69201 |
| | DuDre-Atg8- | | W, Li H, Zhu J, Wang Y, Lin J, Xie | |
| | 404 | | Z. Autophagy. 2015; 11(6): 954-60. doi: | |
| | | | 10.1080/15548627.2015. | |
| pET336 | pMBP-TEV- | XXX | This study | |
| | MCP-6HIS | | | |
| SINAPV5 | Flag- | XXX | Wu et al Science. 2016 | |
| | 24xSuntagV4- | | | |
| | oxBFP-AID- | | | |
| | 24MBSV5 | | | |
| SINAPV6 | Flag- | XXX | This study | |
| | 24xSuntagV4- | | | |
| | oxBFP-AID- | | | |
| | 24MBSV6 | | | |
| pDZ274 | tdTomato | KAN | Larson et al. Science 2011 | |
| | Loxp KanR | | | |
| | Loxp | | | |
| pMC438 | Edc3- | URA3 | Haimovich et al. Cell 2013 | |
| | mCherry | | | |
| | under | | | |
| | endogenous | | | |
| | promoter | | | |
| pRP1152 | Dcp2-RFP | URA3 | Seth and Parker Science 2003 | |
| | under | | | |
| | endogenous | | | |
| | promoter | | | |

TABLE 3
Oligos used in this study (SEQ ID NOS: 13-31, top to bottom, respectively)

| code | Name | Sequence 5′-3′ |
|---|---|---|
| OET156 | ASH1 3UTR Tagging Fw | TGCGAAATTGAAGGGTACCGTTGCTTATTTTGTAATTACATAACTGAGACAGTAGAGAATTGAAACCTAC |
| | (V5) | AAACGGGTGGAGGATCA |
| OET157 | ASH1 3UTR Tagging Fw | TGCGAAATTGAAGGGTACCGTTGCTTATTTTGTAATTACATAACTGAGACAGTAGAGAATTGACCGCTCT |
| | (V6/V7) | AGAACTAGTGGAT |
| OET257 | ASH1 tagging Rev | TGTACAATTGTTTCGTGATAATGTCTCTTATTAGTTGAAAGAGATTCAGTTATCCATGTAGCATAGGCCA |
| | (V5/V6/V7) | CTAGTGGATCTG |
| OET138 | ASH1 3\grave{ }end cds Fw | AACACATACAAGATGTTTGAACG |
| OET214 | Kanr 5\grave{ }end Rev | CTGATTGCCCGACATTATCGC |
| OET258 | DOA1 3UTR tagging Fw | GCTTGCAAACATCAAAAGGAGCTATGGGAACGTGCCAAGGTTTAAGGATATTTTCGACGATCTCTCCTAA |
| | (V6) | CCGCTCTAGAACTAGTGGAT |
| OET259 | DOA1 3UTR tagging | GGGCAGAAAGAATTTTAAAGATTATTTGCTATCTAGACATTATGTGTTTTATATGATTGCTGTAAAAGTA |
| | Rev (V6) | GCATAGGCCACTAGTGGATCTG |
| OET260 | DOA1 3\grave{ }end cds Fw | GGAACGTGCCAAGGTTTAAG |
| MDN1MS2F | MDN1 3UTR tagging Fw | CACGATATAAGCGAACTACCCGAAATGCTTTCACTGATTTTGCGTCAATACTTTACAGACCTGGCATCCA |
| | (V6/V7) | GCTAAcgctctagaactagtggatc |
| MDN1MS2R | MDN1 3UTR tagging | TTTTTTTTCAGTTCCATGATTTTTTTGTTCCTTTGATTCGTGTAGTAAACCTCCTCTTCTTGGTTTTCAC |
| | Rev (V6/V7) | GATATACACTAGTGGATCTGATATCACC |
| SH | MDN1 3UTR tagging Fw | TTCACTGATTTTGCGTCAATACTTTACAGACCTGGCATCCAGCTAAGCCGCTCTAGAACTAGTGGATCC |
| | MS2ORF | |
| SH | MDN1 3UTR tagging | CCTTTGATTCGTGTAGTAAACCTCCTCTTCTTGGTTTTCACGATATAGCATAGGCCACTAGTGGATCTG |
| | Rev MS2ORF | |
| KanChRev | KanChRev | CTGATTGCCCGACATTATCGC |
| MDN1ChF | MDN1ChF | GATGGTATTTGCGAAGACCATG |
| GAL1MS2F | GAL1 3UTR tagging Fw | TGCTGAGCTAGAAAATGCTATCATCGTCTCTAAACCAGCATTGGGCAGCTGTCTATATGAATTATAAGCC |
| | (V6/V7) | CCTGGCAATCGCGGG |
| GAL1MS2Fw | GAL1 3UTR tagging Fw | TTTTGTGATGCTAAAGTTATGAGTAGAAAAAAATGAGAAGTTGTTCTGAACAAAGTAAAAAAAAGAAGT |
| | (V5) | ATACACTAGTGGATCTGATATCACC |
| GAL1MS2R | GAL1 3UTR tagging Rev | TTTTGTGATGCTAAAGTTATGAGTAGAAAAAAATGAGAAGTTGTTCTGAACAAAGTAAAAAAAAGAAGT |
| | (V5/V6/V7) | ATACACTAGTGGATCTGATATCACC |
| GAL1ChF | GAL1ChF | GAAGCCCTTGCCAATGAGTTC |
| JG222 | ASH1 probe for | GTTTCGTGATAATGTCTCTTATTAGTTG |
| | Northern Blot | |

MBSV6_U_variant.
(SEQ ID NO: 2)
TATTCTTTTGATTTATAAGGGATTTTGCCGATTTCGGCCTATTGGTTAAAAAATG
AGCTGATTTAACAAAAATTTAACGCGAATTTTAACAAAATATTAACGCTTACAA
TTTCCATTCGCCATTCAGGCTGCGCAACTGTTGGGAAGGGCGATCGGTGCGGGC
CTCTTCGCTATTACGCCAGCTGGCGAAAGGGGGATGTGCTGCAAGGCGATTAAG
TTGGGTAACGCCAGGGTTTTCCCAGTCACGACGTTGTAAAACGACGGCCAGTGA
GCGCGCGTAATACGACTCACTATAGGGCGAATTGGAGCTCCACCGCGGTGGCG
GCCGCTCTAGAACTAGTGGATCCCAGAGCCCCCTGGCAATCGCGGGGAGACGC
AGGACTACCGCGTCTTCACTCTCGCTTGCGCGGTATACGCAGGGAGCAACGTCA
CCCAGGGCGACACAGAGGAATACCCTGTGTGGCCCCACGGTGCTACAAAGAAC
ATTCCGTATTGCTCACCAGTGTCACATGTGGAGGACTACCCCACAAGCGAGTCA
GGAAACCTTCGGGGCATCGCACCATTATCCCGAACAATCGACACGCAGGATTA
CCGCGTGGGACACTCTGTTCCCCGTCAAAATTGGACCATACCGGAGTCGGCTTA
CGTCATGCAGGATTACCGCATGCATGGTGCAGAATATCGGCATGTCTACTCGTA
CAGTCAAATCTACTCGTGTGGTCAGGACTACCGACCACTTCTATTCTATTCATCT
TTTCGGTTGTGCAGGTATTCTGCCGATGTACGAGAAGACGATTACGCTTCTCGA
CTACCTTCATCATACATGGTGTGCAGATGGCCGCCAAGTTTTTTGCCAATGGAG
GAATACCCCATTCTCTGTCAACCAACCCATGCAAAGTTTACACTCTGCTATGGC
AGCACTGTCGCAGAGGAATACCCTGCGAGCCAAAACGGCCCCCGGTGCGTGTA
TTGCATCTGCCTTGCGAGCATTCACAGGGACGAATACGCCCTGCCGTTGCATTA
CTTCAATATGGGTGCTCTGTCGTCGTCATCAGGACCATTTGCGCAGGACTACCG
CGCATATATCATCAGCACTCGTGCGGATACTTCTGGGATTCCTATTGTTACGCG
AGCTCAGGAATACCGAGCTCTGGCGACAGAGACCCTCACACGgAAGATCTATCG
ATCTCGACAACCCTTAATATAACTTCGTATAATGTATGCTATACGAAGTTATTA
GGTCTAGAGATCTGTTTAGCTTGCCTCGTCCCCGCCGGGTCACCCGGCCAGCGA
CATGGAGGCCCAGAATACCCTCCTTGACAGTCTTGACGTGCGCAGCTCAGGGGC
ATGATGTGACTGTCGCCCGTACATTTAGCCCATACATCCCCATGTATAATCATTT
GCATCCATACATTTTGATGGCCGCACGGCGCGAAGCAAAAATTACGGCTCCTCG
CTGCAGACCTGCGAGCAGGGAAACGCTCCCCTCACAGACGCGTTGAATTGTCCC
CACGCCGCGCCCCTGTAGAGAAATATAAAAGGTTAGGATTTGCCACTGAGGTTC
TTCTTTCATATACTTCCTTTTAAAATCTTGCTAGGATACAGTTCTCACATCACAT
CCGAACATAAACAACCATGGGTAAGGAAAAGACTCACGTTTCGAGGCCGCGAT
TAAATTCCAACATGGATGCTGATTTATATGGGTATAAATGGGCTCGCGATAATG
TCGGGCAATCAGGTGCGACAATCTATCGATTGTATGGGAAGCCCGATGCGCCA
GAGTTGTTTCTGAAACATGGCAAAGGTAGCGTTGCCAATGATGTTACAGATGAG
ATGGTCAGACTAAACTGGCTGACGGAATTTATGCCTCTTCCGACCATCAAGCAT
TTTATCCGTACTCCTGATGATGCATGGTTACTCACCACTGCGATCCCCGGCAAA
ACAGCATTCCAGGTATTAGAAGAATATCCTGATTCAGGTGAAAATATTGTTGAT
GCGCTGGCAGTGTTCCTGCGCCGGTTGCATTCGATTCCTGTTTGTAATTGTCCTT
TTAACAGCGATCGCGTATTTCGTCTCGCTCAGGCGCAATCACGAATGAATAACG
GTTTGGTTGATGCGAGTGATTTTGATGACGAGCGTAATGGCTGGCCTGTTGAAC
AAGTCTGGAAAGAAATGCATAAGCTTTTGCCATTCTCACCGGATTCAGTCGTCA
CTCATGGTGATTTCTCACTTGATAACCTTATTTTTGACGAGGGGAAATTAATAG
GTTGTATTGATGTTGGACGAGTCGGAATCGCAGACCGATACCAGGATCTTGCCA
TCCTATGGAACTGCCTCGGTGAGTTTTCTCCTTCATTACAGAAACGGCTTTTTCA
AAAATATGGTATTGATAATCCTGATATGAATAAATTGCAGTTTCATTTGATGCT
CGATGAGTTTTTCTAATCAGTACTGACAATAAAAAGATTCTTGTTTTCAAGAAC
TTGTCATTTGTATAGTTTTTTTATATTGTAGTTGTTCTATTTTAATCAAATGTTAG
CGTGATTTATATTTTTTTTCGCCTCGACATCATCTGCCCAGATGCGAAGTTAAGT
GCGCAGAAAGTAATATCATGCGTCAATCGTATGTGAATGCTGGTCGCTATACTG
CTGTCGATTCGATACTAACGCCGCCATCCAGTGTCGAAAACGAGCTCTCGAGAA
CCCTTAATATAACTTCGTATAATGTATGCTATACGAAGTTATTAGGTGATATCA
GATCCACTAGTGGCCTATGCGGTACCCAGCTTTTGTTCCCTTTAGTGAGGGTTAA
TTGCGCGCTTGGCGTAATCATGGTCATAGCTGTTTCCTGTGTGAAATTGTTATCC
GCTCACAATTCCACACAACATACGAGCCGGAAGCATAAAGTGTAAAGCCTGGG
GTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGG
GGAGAGGCGGTTTGCGTATTGGGCGCTCTTCCGCTTCCTCGCTCACTGACTCGCT
GCGCTCGGTCGTTCGGCTGCGGCGAGCGGTATCAGCTCACTCAAAGGCGGTAAT
ACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTC
CCTTCGGGAAGCGTGGCGCTTTCTCATAGCTCACGCTGTAGGTATCTCAGTTCG
GTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAG
AAGGACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTT
TGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGA
TTTTGGTCATGAGATTATCAAAAAGGATCTTCACCTAGATCCTTTTAAATTAAA
AATGAAGTTTTAAATCAATCTAAAGTATATATGAGTAAACTTGGTCTGACAGTT
ACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACC
ATCTGGCCCCAGTGCTGCAATGATACCGCGAGACCCACGCTCACCGGCTCCAGA
TTTATCAGCAATAAACCAGCCAGCCGGAAGGGCCGAGCGCAGAAGTGGTCCTG
CAACTTTATCCGCCTCCATCCAGTCTATTAATTGTTGCCGGGAAGCTAGAGTAA
GTAGTTCGCCAGTTAATAGTTTGCGCAACGTTGTTGCCATTGCTACAGGCATCG
TGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGG
TCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTA
AAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCA
GCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTT
CCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCC
CGAAAAGTGCCACCTGACGCGCCCTGTAGCGGCGCATTAAGCGCGGCGGGTGT
GGTGGTTACGCGCAGCGTGACCGCTACACTTGCCAGCGCCCTAGCGCCCGCTCC
TTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACC
CCAAAAAACTTGATTAGGGTGATGGTTCACGTAGTGGGCCATCGCCCTGATAGA
CGGTTTTTCGCCCTTTGACGTTGGAGTCCACGTTCTTTAATAGTGGACTCTTGTT
CCAAACTGGAACAACACTCAACCCTATCTCGGTC
MBSV6_C_variant
(SEQ ID NO: 3)
TATTCTTTTGATTTATAAGGGATTTTGCCGATTTCGGCCTATTGGTTAAAAAATG
AGCTGATTTAACAAAAATTTAACGCGAATTTTAACAAAATATTAACGCTTACAA
TTTCCATTCGCCATTCAGGCTGCGCAACTGTTGGGAAGGGCGATCGGTGCGGGC
CTCTTCGCTATTACGCCAGCTGGCGAAAGGGGGATGTGCTGCAAGGCGATTAAG
TTGGGTAACGCCAGGGTTTTCCCAGTCACGACGTTGTAAAACGACGGCCAGTGA
GCGCGCGTAATACGACTCACTATAGGGCGAATTGGAGCTCCACCGCGGTGGCG
GCCGCTCTAGAACTAGTGGATCCCAGAGCCCCCTGGCAATCGCGGGGAGACGC
AGGACcACCGCGTCTTCACTCTCGCTTGCGCGGTATACGCAGGGAGCAACGTCA
CCCAGGGCGACACAGAGGAAcACCCTGTGTGGCCCCACGGTGCTACAAAGAAC
ATTCCGTATTGCTCACCAGTGTCACATGTGGAGGACcACCCCACAAGCGAGTCA
GGAAACCTTCGGGGCATCGCACCATTATCCCGAACAATCGACACGCAGGATcAC
CGCGTGGGACACTCTGTTCCCCGTCAAAATTGGACCATACCGGAGTCGGCTTAC
GTCATGCAGGATcACCGCATGCATGGTGCAGAATATCGGCATGTCTACTCGTAC
AGTCAAATCTACTCGTGTGGTCAGGACcACCGACCACTTCTATTCTATTCATCTT
TTCGGTTGTGCAGGTATTCTGCCGATGTACGAGAAGACGATcACGCTTCTCGACT
ACCTTCATCATACATGGTGTGCAGATGGCCGCCAAGTTTTTTGCCAATGGAGGA
AcACCCCATTCTCTGTCAACCAACCCATGCAAAGTTTACACTCTGCTATGGCAGC
ACTGTCGCAGAGGAAcACCCTGCGAGCCAAAACGGCCCCCGGTGCGTGTATTGC
ATCTGCCTTGCGAGCATTCACAGGGACAAcACGCCCTGCCGTTGCATTACTTC
AATATGGGTGCTCTGTCGTCGTCATCAGGACCATTTGCGCAGGACcACCGCGCA
TATATCATCAGCACTCGTGCGGATACTTCTGGGATTCCTATTGTTACGCGAGCTC
AGGAAcACCGAGCTCTGGCGACAGAGACCCTCACACGgAAGATCTATCGATCTC
GACAACCCTTAATATAACTTCGTATAATGTATGCTATACGAAGTTATTAGGTCT
AGAGATCTGTTTAGCTTGCCTCGTCCCCGCCGGGTCACCCGGCCAGCGACATGG
AGGCCCAGAATACCCTCCTTGACAGTCTTGACGTGCGCAGCTCAGGGGCATGAT
GTGACTGTCGCCCGTACATTTAGCCCATACATCCCCATGTATAATCATTTGCATC
CATACATTTTGATGGCCGCACGGCGCGAAGCAAAAATTACGGCTCCTCGCTGCA
GACCTGCGAGCAGGGAAACGCTCCCCTCACAGACGCGTTGAATTGTCCCCACGC
CGCGCCCCTGTAGAGAAATATAAAAGGTTAGGATTTGCCACTGAGGTTCTTCTT
TCATATACTTCCTTTTAAAATCTTGCTAGGATACAGTTCTCACATCACATCCGAA
CATAAACAACCATGGGTAAGGAAAAGACTCACGTTTCGAGGCCGCGATTAAAT
TCCAACATGGATGCTGATTTATATGGGTATAAATGGGCTCGCGATAATGTCGGG
CAATCAGGTGCGACAATCTATCGATTGTATGGGAAGCCCGATGCGCCAGAGTTG
TTTCTGAAACATGGCAAAGGTAGCGTTGCCAATGATGTTACAGATGAGATGGTC
AGACTAAACTGGCTGACGGAATTTATGCCTCTTCCGACCATCAAGCATTTTATC
CGTACTCCTGATGATGCATGGTTACTCACCACTGCGATCCCCGGCAAAACAGCA
TTCCAGGTATTAGAAGAATATCCTGATTCAGGTGAAAATATTGTTGATGCGCTG
GCAGTGTTCCTGCGCCGGTTGCATTCGATTCCTGTTTGTAATTGTCCTTTTAACA
GCGATCGCGTATTTCGTCTCGCTCAGGCGCAATCACGAATGAATAACGGTTTGG
TTGATGCGAGTGATTTTGATGACGAGCGTAATGGCTGGCCTGTTGAACAAGTCT
GGAAAGAAATGCATAAGCTTTTGCCATTCTCACCGGATTCAGTCGTCACTCATG
GTGATTTCTCACTTGATAACCTTATTTTTGACGAGGGGAAATTAATAGGTTGTAT
TGATGTTGGACGAGTCGGAATCGCAGACCGATACCAGGATCTTGCCATCCTATG
GAACTGCCTCGGTGAGTTTTCTCCTTCATTACAGAAACGGCTTTTTCAAAAATAT
GGTATTGATAATCCTGATATGAATAAATTGCAGTTTCATTTGATGCTCGATGAG
TTTTTCTAATCAGTACTGACAATAAAAAGATTCTTGTTTTCAAGAACTTGTCATT
TGTATAGTTTTTTTATATTGTAGTTGTTCTATTTTAATCAAATGTTAGCGTGATTT
ATATTTTTTTTCGCCTCGACATCATCTGCCCAGATGCGAAGTTAAGTGCGCAGA
AAGTAATATCATGCGTCAATCGTATGTGAATGCTGGTCGCTATACTGCTGTCGA
TTCGATACTAACGCCGCCATCCAGTGTCGAAAACGAGCTCTCGAGAACCCTTAA
TATAACTTCGTATAATGTATGCTATACGAAGTTATTAGGTGATATCAGATCCAC
TAGTGGCCTATGCGGTACCCAGCTTTTGTTCCCTTTAGTGAGGGTTAATTGCGCG
CTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTA
ATGAGTGAGCTAACTCACATTAATTGCGTTGCGCTCACTGCCCGCTTTCCAGTC
GGGAAACCTGTCGTGCCAGCTGCATTAATGAATCGGCCAACGCGCGGGGAGAG
GCGGTTTGCGTATTGGGCGCTCTTCCGCTTCCTCGCTCACTGACTCGCTGCGCTC
GGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCCCTTCG
GGAAGCGTGGCGCTTTCTCATAGCTCACGCTGTAGGTATCTCAGTTCGGTGTAG
GTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTA
TCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGGAC
AGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAG
TTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGC
CCCAGTGCTGCAATGATACCGCGAGACCCACGCTCACCGGCTCCAGATTTATCA
GCAATAAACCAGCCAGCCGGAAGGGCCGAGCGCAGAAGTGGTCCTGCAACTTT
ATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAAGGCG
AGTTACATGATCCCCCATGTTGTGCAAAAAAGCGGTTAGCTCCTTCGGTCCTCC
GATCGTTGTCAGAAGTAAGTTGGCCGCAGTGTTATCACTCATGGTTATGGCAGC
ACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCT
TGCCCGGCGTCAATACGGGATAATACCGCGCCACATAGCAGAACTTTAAAAGT
GCTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGC
AAAAAAGGGAATAAGGGCGACACGGAAATGTTGAATACTCATACTCTTCCTTTT
TCAATATTATTGAAGCATTTATCAGGGTTATTGTCTCATGAGCGGATACATATTT
GAATGTATTTAGAAAAATAAACAAATAGGGGTTCCGCGCACATTTCCCCGAAA
AGTGCCACCTGACGCGCCCTGTAGCGGCGCATTAAGCGCGGCGGGTGTGGTGG
TTACGCGCAGCGTGACCGCTACACTTGCCAGCGCCCTAGCGCCCGCTCCTTTCG
CTTTCTTCCCTTCCTTTCTCGCCACGTTCGCCGGCTTTCCCCGTCAAGCTCTAAAT
CGGGGGCTCCCTTTAGGGTTCCGATTTAGTGCTTTACGGCACCTCGACCCCAAA
AAACTTGATTAGGGTGATGGTTCACGTAGTGGGCCATCGCCCTGATAGACGGTT
TTTCGCCCTTTGACGTTGGAGTCCACGTTCTTTAATAGTGGACTCTTGTTCCAAA
CTGGAACAACACTCAACCCTATCTCGGTC
YcpLac111CYC1p_MCP_NLS_2xGFP
(SEQ ID NO: 4)
TACGCAAACCGCCTCTCCCCGCGCGTTGGCCGATTCATTAATGCAGCTGGCACG
ACAGGTTTCCCGACTGGAAAGCGGGCAGTGAGCGCAACGCAATTAATGTGAGT
TAGCTCACTCATTAGGCACCCCAGGCTTTACACTTTATGCTTCCGGCTCGTATGT
TGTGTGGAATTGTGAGCGGATAACAATTTCACACAGGAAACAGCTATGACCAT
GATTACGCCAAGCTTGCATGCCTGCAGGGAGCGTTGGTTGGTGGATCAAGCCCA
CGCGTAGGCAATCCTCGAGCAGATCCGCCGGGCGTGTATATAGCGTGGATGGC
CAGGCAACTTTAGTGCTGACACATACAGGCATATATATATGTGTGCGACGACAC
ATGATCATATGGCATGCATGTGCTCTGTATGTATATAAAACTCTTGTTTTCTTCT
TTTCTCTAAATATTCTTTCCTTATACATTAGGTCCTTTGTAGCATAAATTACTATA
CTTCTATAGACACGCAAACACAAATACACACACTAAATTAATAGGATCCATGCT
AGCCGTTAAAATGGCTTCTAACTTTACTCAGTTCGTTCTCGTCGACAATGGCGG
AACTGGCGACGTGACTGTCGCCCCAAGCAACTTCGCTAACGGGATCGCTGAATG
GATCAGCTCTAACTCGCGTTCACAGGCTTACAAAGTAACCTGTAGCGTTCGTCA
GAGCTCTGCGCAGAATCGCAAATACACCATCAAAGTCGAGGTGCCTAAAGGCG
CCTGGCGTTCGTACTTAAATATGGAACTAACCATTCCAATTTTCGCCACGAATTC
CGACTGCGAGCTTATTGTTAAGGCAATGCAAGGTCTCCTAAAAGATGGAAACCC
GATTCCCTCAGCAATCGCAGCAAACTCCGGCATCTACCCAAAAAAAAAAAGAA
AAGTTACCGGTTCTAAAGGTGAAGAATTATTCACTGGTGTTGTCCCAATTTTGG
TTGAATTAGATGGTGATGTTAATGGTCACAAATTTTCTGTCTCCGGTGAAGGTG
AAGGTGATGCTACTTACGGTAAATTGACCTTAAAATTTATTTGTACTACTGGTA
AATTGCCAGTTCCATGGCCAACCTTAGTCACTACTTTAACTTATGGTGTTCAATG
TTTTTCTAGATACCCAGATCATATGAAACAACATGACTTTTTCAAGTCTGCCATG
CCAGAAGGTTATGTTCAAGAAAGAACTATTTTTTTCAAAGATGACGGTAACTAC
AAGACCAGAGCTGAAGTCAAGTTTGAAGGTGATACCTTAGTTAATAGAATCAA
ATTAAAAGGTATTGATTTTAAAGAAGATGGTAACATTTTAGGTCACAAATTGGA
ATACAACTATAACTCTCACAATGTTTACATCATGGCTGACAAACAAAAGAATGG
TATCAAAGTTAACTTCAAAATTAGACACAACATTGAAGATGGTTCTGTTCAATT
AGCTGACCATTATCAACAAAATACTCCAATTGGTGATGGTCCAGTCTTGTTACC
AGACAACCATTACTTATCCACTCAATCTGCCTTATCCAAAGATCCAAACGAAAA
GAGAGACCACATGGTCTTGTTAGAATTTGTTACTGCTGCTGGTATTACCCATGG
TATGGATGAATTGTACAAATGTTTAAACTCTAAAGGTGAAGAATTATTCACTGG
TGTTGTCCCAATTTTGGTTGAATTAGATGGTGATGTTAATGGTCACAAATTTTCT
GTCTCCGGTGAAGGTGAAGGTGATGCTACTTACGGTAAATTGACCTTAAAATTT
ATTTGTACTACTGGTAAATTGCCAGTTCCATGGCCAACCTTAGTCACTACTTTAA
CTTATGGTGTTCAATGTTTTTCTAGATACCCAGATCATATGAAACAACATGACTT
TTTCAAGTCTGCCATGCCAGAAGGTTATGTTCAAGAAAGAACTATTTTTTTCAA
AGATGACGGTAACTACAAGACCAGAGCTGAAGTCAAGTTTGAAGGTGATACCT
TAGTTAATAGAATCGAATTAAAAGGTATTGATTTTAAAGAAGATGGTAACATTT
TAGGTCACAAATTGGAATACAACTATAACTCTCACAATGTTTACATCATGGCTG
ACAAACAAAAGAATGGTATCAAAGTTAACTTCAAAATTAGACACAACATTGAA
GATGGTTCTGTTCAATTAGCTGACCATTATCAACAAAATACTCCAATTGGTGAT
GGTCCAGTCTTGTTACCAGACAACCATTACTTATCCACTCAATCTGCCTTATCCA
AAGATCCAAACGAAAAGAGAGACCACATGGTCTTGTTAGAATTTGTTACTGCTG
CTGGTATTACCCATGGTATGGATGAATTGTACAAATAAGTTTAAACCCGCTGAT
CCTAGAGGGCCGCATCATGTAATTAGTTATGTCACGCTTACATTCACGCCCTCC
CCCCACATCCGCTCTAACCGAAAAGGAAGGAGTTAGACAACCTGAAGTCTAGG
TCCCTATTTATTTTTTTATAGTTATGTTAGTATTAAGAACGTTATTTATATTTCAA
ATTTTTCTTTTTTTTCTGTACAGACGCGTGTACGCATGTAACATTATACTGAAAA
CCTTGCTTGAGAAGGTTTTGGGACGCTCGAAGGCTTTAATTTGAGCTCGAATTC
ACTGGCCGTCGTTTTACAACGTCGTGACTGGGAAAACCCTGGCGTTACCCAACT
TAATCGCCTTGCAGCACATCCCCCTTTCGCCAGCTGGCGTAATAGCGAAGAGGC
CCGCACCGATCGCCCTTCCCAACAGTTGCGCAGCCTGAATGGCGAATGGCGCCT
GATGCGGTATTTTCTCCTTACGCATCTGTGCGGTATTTCACACCGCATAATCGCT
GGGCCATTCTCATGAAGAATATCTTGAATTTATTGTCATATTACTAGTTGGTGTG
GAAGTCCTAATATCGGTGATCAATATAGTGGTTGACATGCTGGCTAGTCAACAT
TGAGCCTTTTGATCATGCAAATATATTACGGTATTTTACAATCAAATATCAAACT
TAACTATTGACTTTATAACTTATTTAGGTGGTAACATTCTTATAAAAAAGAAAA
AAATTACTGCAAAACAGTACTAGCTTTTAACTTGTATCCTAGGTTATCTATGCTG
TCTCACCATAGAGAATATTACCTATTTCAGAATGTATGTCCATGATTCGCCGGG
TAAATACATATAATACACAAATCTGGCTTAATAAAGTCTATAATATATCTCATA
AAGAAGTGCTAAATTGGCTAGTGCTATATATTTTTAAGAAAATTTCTTTTGACTA
AGTCCATATCGACTTTGTAAAAGTTCACATTAGCATACATATATTACACGAGCC
AGAAATAGTAACTTTTGCCTAAATCACAAATTGCAAAATTTAATTGCTTGCAAA
AGGTCACATGCTTATAATCAACTTTTTTAAAAATTTAAAATACTTTTTTATTTTTT
ATTTTTAAACATAAATGAAATAATTTATTTATTGTTTATGATTACCGAAACATAA
AACCTGCTCAAGAAAAAGAAACTGTTTTGTCCTTGGAAAAAAAGCACTACCTA
GGAGCGGCCAAAATGCCGAGGCTTTCATAGCTTAAACTCTTTACAGAAAATAG
GCATTATAGATCAGTTCGAGTTTTCTTATTCTTCCTTCCGGTTTTATCGTCACAGT
TTTACAGTAAATAAGTATCACCTCTTAGAGTTCGATGATAAGCTGTCAAACATG
AGAATTAATTCCACATGTTAAAATAGTGAAGGAGCATGTTCGGCACACAGTGG
ACCGAACGTGGGGTAAGTGCACTAGGGTCCGGTTAAACGGATCTCGCATTGAT
GAGGCAACGCTAATTATCAACATATAGATTGTTATCTATCTGCATGAACACGAA
ATCTTTACTTGACGACTTGAGGCTGATGGTGTTTATGCAAAGAAACCACTGTGT
TTAATATGTGTCACTGTTTGATATTACTGTCAGCGTAGAAGATAATAGTAAAAG
CGGTTAATAAGTGTATTTGAGATAAGTGTGATAAAGTTTTTACAGCGAAAAGAC
GATAAATACAAGAAAATGATTACGAGGATACGGAGAGAGGTATGTACATGTGT
ATTTATATACTAAGCTGCCGGCGGTTGTTTGCAAGACCGAGAAAAGGCTAGCAA
GAATCGGGTCATTGTAGCGTATGCGCCTGTGAACATTCTCTTCAACAAGTTTGA
TTCCATTGCGGTGAAATGGTAAAAGTCAACCCCCTGCGATGTATATTTTCCTGT
ACAATCAATCAAAAAGCCAAATGATTTAGCATTATCTTTACATCTTGTTATTTTA
CAGATTTTATGTTTAGATCTTTTATGCTTGCTTTTCAAAAGGCTTGCAGGCAAGT
GCACAAACAATACTTAAATAAATACTACTCAGTAATAACCTATTTCTTAGCATT
TTTGACGAAATTTGCTATTTTGTTAGAGTCTTTTACACCATTTGTCTCCACACCT
CCGCTTACATCAACACCAATAACGCCATTTAATCTAAGCGCATCACCAACATTT
TCTGGCGTCAGTCCACCAGCTAACATAAAATGTAAGCTCTCGGGGCTCTCTTGC
CTTCCAACCCAGTCAGAAATCGAGTTCCAATCCAAAAGTTCACCTGTCCCACCT
GCTTCTGAATCAAACAAGGGAATAAACGAATGAGGTTTCTGTGAAGCTGCACT
GAGTAGTATGTTGCAGTCTTTTGGAAATACGAGTCTTTTAATAACTGGCAAACC
GAGGAACTCTTGGTATTCTTGCCACGACTCATCTCCATGCAGTTGGACGATCGA
TGATAAGCTGTCAAACATGAGAATTAATTCTACCCTATGAACATATTCCATTTT
GTAATTTCGTGTCGTTTCTATTATGAATTTCATTTATAAAGTTTATGTACAAATA
TCATAAAAAAAGAGAATCTTTTTAAGCAAGGATTTTCTTAACTTCTTCGGCGAC
AGCATCACCGACTTCGGTGGTACTGTTGGAACCACCTAAATCACCAGTTCTGAT
ACCTGCATCCAAAACCTTTTTAACTGCATCTTCAATGGCCTTACCTTCTTCAGGC
AAGTTCAATGACAATTTCAACATCATTGCAGCAGACAAGATAGTGGCGATAGG
GTTGACCTTATTCTTTGGCAAATCTGGAGCAGAACCGTGGCATGGTTCGTACAA
ACCAAATGCGGTGTTCTTGTCTGGCAAAGAGGCCAAGGACGCAGATGGCAACA
AACCCAAGGAACCTGGGATAACGGAGGCTTCATCGGAGATGATATCACCAAAC
ATGTTGCTGGTGATTATAATACCATTTAGGTGGGTTGGGTTCTTAACTAGGATC
ATGGCGGCAGAATCAATCAATTGATGTTGAACCTTCAATGTAGGAAATTCGTTC
TTGATGGTTTCCTCCACAGTTTTTCTCCATAATCTTGAAGAGGCCAAAACATTAG
CTTTATCCAAGGACCAAATAGGCAATGGTGGCTCATGTTGTAGGGCCATGAAAG
CGGCCATTCTTGTGATTCTTTGCACTTCTGGAACGGTGTATTGTTCACTATCCCA
AGCGACACCATCACCATCGTCTTCCTTTCTCTTACCAAAGTAAATACCTCCCACT
AATTCTCTGACAACAACGAAGTCAGTACCTTTAGCAAATTGTGGCTTGATTGGA
GATAAGTCTAAAAGAGAGTCGGATGCAAAGTTACATGGTCTTAAGTTGGCGTA
CAATTGAAGTTCTTTACGGATTTTTAGTAAACCTTGTTCAGGTCTAACACTACCT
GTACCCCATTTAGGACCACCCACAGCACCTAACAAAACGGCATCAGCCTTCTTG
GAGGCTTCCAGCGCCTCATCTGGAAGTGGAACACCTGTAGCATCGATAGCAGC
ACCACCAATTAAATGATTTTCGAAATCGAACTTGACATTGGAACGAACATCAGA
AATAGCTTTAAGAACCTTAATGGCTTCGGCTGTGATTTCTTGACCAACGTGGTC
ACCTGGCAAAACGACGATCTTCTTAGGGGCAGACATTAGAATGGTATATCCTTG
AAATATATATATATATTGCTGAAATGTAAAAGGTAAGAAAAGTTAGAAAGTAA
GACGATTGCTAACCACCTATTGGAAAAAACAATAGGTCCTTAAATAATATTGTC
AACTTCAAGTATTGTGATGCAAGCATTTAGTCATGAACGCTTCTCTATTCTATAT
GAAAAGCCGGTTCCGGCGCTCTCACCTTTCCTTTTTCTCCCAATTTTTCAGTTGA
AAAAGGTATATGCGTCAGGCGACCTCTGAAATTAACAAAAAATTTCCAGTCATC
GAATTTGATTCTGTGCGATAGCGCCCCTGTGTGTTCTCGTTATGTTGAGGAAAA
AAATAATGGTTGCTAAGAGATTCGAACTCTTGCATCTTACGATACCTGAGTATT
CCCACAGTTAATTCTTGAAGACGAAAGGGCCTCGTGATACGCCTATTTTTATAG
GTTAATGTCATGATAATAATGGTTTCTTAGACGTCAGGTGGCACTTTTCGGGGA
AATGTGCGCGGAACCCCTATTTGTTTATTTTTCTAAATACATTCAAATATGTATC
CGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTT
GCCTTCCTGTTTTTGCTCACCCAGAAACGCTGGTGAAAGTAAAAGATGCTGAAG
ATCAGTTGGGTGCACGAGTGGGTTACATCGAACTGGATCTCAACAGCGGTAAG
ATCCTTGAGAGTTTTCGCCCCGAAGAACGTTTTCCAATGATGAGCACTTTTAAA
GTTCTGCTATGTGGCGCGGTATTATCCCGTATTGACGCCGGGCAAGAGCAACTC
GGTCGCCGCATACACTATTCTCAGAATGACTTGGTTGAGTACTCACCAGTCACA
GAAAAGCATCTTACGGATGGCATGACAGTAAGAGAATTATGCAGTGCTGCCAT
AACCATGAGTGATAACACTGCGGCCAACTTACTTCTGACAACGATCGGAGGAC
CGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACC
ACGATGCCTGTAGCAATGGCAACAACGTTGCGCAAACTATTAACTGGCGAACT
ACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAA
ATCTGGAGCCGGTGAGCGTGGGTCTCGCGGTATCATTGCAGCACTGGGGCCAG
ATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCAT
TGGTAACTGTCAGACCAAGTTTACTCATATATACTTTAGATTGATTTAAAACTTC
ATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACAC
CGAACTGAGATACCTACAGCGTGAGCTATGAGAAAGCGCCACGCTTCCCGAAG
GGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTT
TCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGT
ATTACCGCCTTTGAGTGAGCTGATACCGCTCGCCGCAGCCGAACGACCGAGCGC
AGCGAGTCAGTGAGCGAGGAAGCGGAAGAGCGCCCAA
(SEQ ID NO: 32)
TACGCAAACCGCCTCTCCCCGCGCGTTGGCCGATTCATTAATGCAGCT
GGCACGACAGGTTTCCCGACTGGAAAGCGGGCAGTGAGCGCAACGCAATTAAT
GTGAGTTAGCTCACTCATTAGGCACCCCAGGCTTTACACTTTATGCTTCCGGCTC
GTATGTTGTGTGGAATTGTGAGCGGATAACAATTTCACACAGGAAACAGCTATG
ACCATGATTACGCCAAGCTTGCATGCCTGCAGGGAGCGTTGGTTGGTGGATCAA
GCCCACGCGTAGGCAATCCTCGAGCAGATCCGCCGGGCGTGTATATAGCGTGG
ATGGCCAGGCAACTTTAGTGCTGACACATACAGGCATATATATATGTGTGCGAC
GACACATGATCATATGGCATGCATGTGCTCTGTATGTATATAAAACTCTTGTTTT
CTTCTTTTCTCTAAATATTCTTTCCTTATACATTAGGTCCTTTGTAGCATAAATTA
CTATACTTCTATAGACACGCAAACACAAATACACACACTAAATTAATAGGATCC
ATGCTAGCCGTTAAAATGGCTTCTAACTTTACTCAGTTCGTTCTCGTCGACAATG
GCGGAACTGGCGACGTGACTGTCGCCCCAAGCAACTTCGCTAACGGGATCGCT
GAATGGATCAGCTCTAACTCGCGTTCACAGGCTTACAAAGTAACCTGTAGCGTT
CGTCAGAGCTCTGCGCAGAATCGCAAATACACCATCAAAGTCGAGGTGCCTAA
AGGCGCCTGGCGTTCGTACTTAAATATGGAACTAACCATTCCAATTTTCGCCAC
GAATTCCGACTGCGAGCTTATTGTTAAGGCAATGCAAGGTCTCCTAAAAGATGG
AAACCCGATTCCCTCAGCAATCGCAGCAAACTCCGGCATCTACACCGGTTCTAA
AGGTGAAGAATTATTCACTGGTGTTGTCCCAATTTTGGTTGAATTAGATGGTGA
TGTTAATGGTCACAAATTTTCTGTCTCCGGTGAAGGTGAAGGTGATGCTACTTA
CGGTAAATTGACCTTAAAATTTATTTGTACTACTGGTAAATTGCCAGTTCCATGG
CCAACCTTAGTCACTACTTTAACTTATGGTGTTCAATGTTTTTCTAGATACCCAG
ATCATATGAAACAACATGACTTTTTCAAGTCTGCCATGCCAGAAGGTTATGTTC
AAGAAAGAACTATTTTTTTCAAAGATGACGGTAACTACAAGACCAGAGCTGAA
GTCAAGTTTGAAGGTGATACCTTAGTTAATAGAATCAAATTAAAAGGTATTGAT
TTTAAAGAAGATGGTAACATTTTAGGTCACAAATTGGAATACAACTATAACTCT
CACAATGTTTACATCATGGCTGACAAACAAAAGAATGGTATCAAAGTTAACTTC
AAAATTAGACACAACATTGAAGATGGTTCTGTTCAATTAGCTGACCATTATCAA
CAAAATACTCCAATTGGTGATGGTCCAGTCTTGTTACCAGACAACCATTACTTA
TCCACTCAATCTGCCTTATCCAAAGATCCAAACGAAAAGAGAGACCACATGGTC
TTGTTAGAATTTGTTACTGCTGCTGGTATTACCCATGGTATGGATGAATTGTACA
AATGTTTAAACTCTAAAGGTGAAGAATTATTCACTGGTGTTGTCCCAATTTTGGT
TGAATTAGATGGTGATGTTAATGGTCACAAATTTTCTGTCTCCGGTGAAGGTGA
AGGTGATGCTACTTACGGTAAATTGACCTTAAAATTTATTTGTACTACTGGTAA
ATTGCCAGTTCCATGGCCAACCTTAGTCACTACTTTAACTTATGGTGTTCAATGT
TTTTCTAGATACCCAGATCATATGAAACAACATGACTTTTTCAAGTCTGCCATGC
CAGAAGGTTATGTTCAAGAAAGAACTATTTTTTTCAAAGATGACGGTAACTACA
AGACCAGAGCTGAAGTCAAGTTTGAAGGTGATACCTTAGTTAATAGAATCGAA
TTAAAAGGTATTGATTTTAAAGAAGATGGTAACATTTTAGGTCACAAATTGGAA
TACAACTATAACTCTCACAATGTTTACATCATGGCTGACAAACAAAAGAATGGT
ATCAAAGTTAACTTCAAAATTAGACACAACATTGAAGATGGTTCTGTTCAATTA
GCTGACCATTATCAACAAAATACTCCAATTGGTGATGGTCCAGTCTTGTTACCA
GACAACCATTACTTATCCACTCAATCTGCCTTATCCAAAGATCCAAACGAAAAG
AGAGACCACATGGTCTTGTTAGAATTTGTTACTGCTGCTGGTATTACCCATGGT
ATGGATGAATTGTACAAATACCCAAAAAAAAAAAGAAAAGTTtaaGTTTAAACC
CGCTGATCCTAGAGGGCCGCATCATGTAATTAGTTATGTCACGCTTACATTCAC
GCCCTCCCCCCACATCCGCTCTAACCGAAAAGGAAGGAGTTAGACAACCTGAA
GTCTAGGTCCCTATTTATTTTTTTATAGTTATGTTAGTATTAAGAACGTTATTTAT
ATTTCAAATTTTTCTTTTTTTTCTGTACAGACGCGTGTACGCATGTAACATTATA
CTGAAAACCTTGCTTGAGAAGGTTTTGGGACGCTCGAAGGCTTTAATTTGCAAG
CTGCGGCCCTGCATTGGCGCGTGTACGCATGTAACATTATACTGAAAACCTTGC
TTGAGAAGGTTTTGGGACGCTCGAAGGCTTTAATTTGAGCTCGAATTCACTGGC
CGTCGTTTTACAACGTCGTGACTGGGAAAACCCTGGCGTTACCCAACTTAATCG
CCTTGCAGCACATCCCCCTTTCGCCAGCTGGCGTAATAGCGAAGAGGCCCGCAC
CGATCGCCCTTCCCAACAGTTGCGCAGCCTGAATGGCGAATGGCGCCTGATGCG
GTATTTTCTCCTTACGCATCTGTGCGGTATTTCACACCGCATAATCGCTGGGCCA
TTCTCATGAAGAATATCTTGAATTTATTGTCATATTACTAGTTGGTGTGGAAGTC
CTAATATCGGTGATCAATATAGTGGTTGACATGCTGGCTAGTCAACATTGAGCC
TTTTGATCATGCAAATATATTACGGTATTTTACAATCAAATATCAAACTTAACTA
TTGACTTTATAACTTATTTAGGTGGTAACATTCTTATAAAAAAGAAAAAAATTA
CTGCAAAACAGTACTAGCTTTTAACTTGTATCCTAGGTTATCTATGCTGTCTCAC
CATAGAGAATATTACCTATTTCAGAATGTATGTCCATGATTCGCCGGGTAAATA
CATATAATACACAAATCTGGCTTAATAAAGTCTATAATATATCTCATAAAGAAG
TGCTAAATTGGCTAGTGCTATATATTTTTAAGAAAATTTCTTTTGACTAAGTCCA
TATCGACTTTGTAAAAGTTCACATTAGCATACATATATTACACGAGCCAGAAAT
AGTAACTTTTGCCTAAATCACAAATTGCAAAATTTAATTGCTTGCAAAAGGTCA
CATGCTTATAATCAACTTTTTTAAAAATTTAAAATACTTTTTTATTTTTTATTTTT
AAACATAAATGAAATAATTTATTTATTGTTTATGATTACCGAAACATAAAACCT
GCTCAAGAAAAAGAAACTGTTTTGTCCTTGGAAAAAAAGCACTACCTAGGAGC
GGCCAAAATGCCGAGGCTTTCATAGCTTAAACTCTTTACAGAAAATAGGCATTA
TAGATCAGTTCGAGTTTTCTTATTCTTCCTTCCGGTTTTATCGTCACAGTTTTACA
GTAAATAAGTATCACCTCTTAGAGTTCGATGATAAGCTGTCAAACATGAGAATT
AATTCCACATGTTAAAATAGTGAAGGAGCATGTTCGGCACACAGTGGACCGAA
CGTGGGGTAAGTGCACTAGGGTCCGGTTAAACGGATCTCGCATTGATGAGGCA
ACGCTAATTATCAACATATAGATTGTTATCTATCTGCATGAACACGAAATCTTT
ACTTGACGACTTGAGGCTGATGGTGTTTATGCAAAGAAACCACTGTGTTTAATA
TGTGTCACTGTTTGATATTACTGTCAGCGTAGAAGATAATAGTAAAAGCGGTTA
ATAAGTGTATTTGAGATAAGTGTGATAAAGTTTTTACAGCGAAAAGACGATAA
ATACAAGAAAATGATTACGAGGATACGGAGAGAGGTATGTACATGTGTATTTA
TATACTAAGCTGCCGGCGGTTGTTTGCAAGACCGAGAAAAGGCTAGCAAGAAT
CGGGTCATTGTAGCGTATGCGCCTGTGAACATTCTCTTCAACAAGTTTGATTCCA
TTGCGGTGAAATGGTAAAAGTCAACCCCCTGCGATGTATATTTTCCTGTACAAT
CAATCAAAAAGCCAAATGATTTAGCATTATCTTTACATCTTGTTATTTTACAGAT
TTTATGTTTAGATCTTTTATGCTTGCTTTTCAAAAGGCTTGCAGGCAAGTGCACA
AACAATACTTAAATAAATACTACTCAGTAATAACCTATTTCTTAGCATTTTTGAC
GAAATTTGCTATTTTGTTAGAGTCTTTTACACCATTTGTCTCCACACCTCCGCTT
ACATCAACACCAATAACGCCATTTAATCTAAGCGCATCACCAACATTTTCTGGC
GTCAGTCCACCAGCTAACATAAAATGTAAGCTCTCGGGGCTCTCTTGCCTTCCA
ACCCAGTCAGAAATCGAGTTCCAATCCAAAAGTTCACCTGTCCCACCTGCTTCT
GAATCAAACAAGGGAATAAACGAATGAGGTTTCTGTGAAGCTGCACTGAGTAG
TATGTTGCAGTCTTTTGGAAATACGAGTCTTTTAATAACTGGCAAACCGAGGAA
CTCTTGGTATTCTTGCCACGACTCATCTCCATGCAGTTGGACGATCGATGATAA
GCTGTCAAACATGAGAATTAATTCTACCCTATGAACATATTCCATTTTGTAATTT
CGTGTCGTTTCTATTATGAATTTCATTTATAAAGTTTATGTACAAATATCATAAA
AAAAGAGAATCTTTTTAAGCAAGGATTTTCTTAACTTCTTCGGCGACAGCATCA
CCGACTTCGGTGGTACTGTTGGAACCACCTAAATCACCAGTTCTGATACCTGCA
TCCAAAACCTTTTTAACTGCATCTTCAATGGCCTTACCTTCTTCAGGCAAGTTCA
ATGACAATTTCAACATCATTGCAGCAGACAAGATAGTGGCGATAGGGTTGACCT
TATTCTTTGGCAAATCTGGAGCAGAACCGTGGCATGGTTCGTACAAACCAAATG
CGGTGTTCTTGTCTGGCAAAGAGGCCAAGGACGCAGATGGCAACAAACCCAAG
GAACCTGGGATAACGGAGGCTTCATCGGAGATGATATCACCAAACATGTTGCT
GGTGATTATAATACCATTTAGGTGGGTTGGGTTCTTAACTAGGATCATGGCGGC
AGAATCAATCAATTGATGTTGAACCTTCAATGTAGGAAATTCGTTCTTGATGGT
TTCCTCCACAGTTTTTCTCCATAATCTTGAAGAGGCCAAAACATTAGCTTTATCC
AAGGACCAAATAGGCAATGGTGGCTCATGTTGTAGGGCCATGAAAGCGGCCAT
TCTTGTGATTCTTTGCACTTCTGGAACGGTGTATTGTTCACTATCCCAAGCGACA
CCATCACCATCGTCTTCCTTTCTCTTACCAAAGTAAATACCTCCCACTAATTCTC
TGACAACAACGAAGTCAGTACCTTTAGCAAATTGTGGCTTGATTGGAGATAAGT
CTAAAAGAGAGTCGGATGCAAAGTTACATGGTCTTAAGTTGGCGTACAATTGA
AGTTCTTTACGGATTTTTAGTAAACCTTGTTCAGGTCTAACACTACCTGTACCCC
ATTTAGGACCACCCACAGCACCTAACAAAACGGCATCAGCCTTCTTGGAGGCTT
CCAGCGCCTCATCTGGAAGTGGAACACCTGTAGCATCGATAGCAGCACCACCA
ATTAAATGATTTTCGAAATCGAACTTGACATTGGAACGAACATCAGAAATAGCT
TTAAGAACCTTAATGGCTTCGGCTGTGATTTCTTGACCAACGTGGTCACCTGGC
AAAACGACGATCTTCTTAGGGGCAGACATTAGAATGGTATATCCTTGAAATATA
TATATATATTGCTGAAATGTAAAAGGTAAGAAAAGTTAGAAAGTAAGACGATT
GCTAACCACCTATTGGAAAAAACAATAGGTCCTTAAATAATATTGTCAACTTCA
AGTATTGTGATGCAAGCATTTAGTCATGAACGCTTCTCTATTCTATATGAAAAG
CCGGTTCCGGCGCTCTCACCTTTCCTTTTTCTCCCAATTTTTCAGTTGAAAAAGG
TATATGCGTCAGGCGACCTCTGAAATTAACAAAAAATTTCCAGTCATCGAATTT
GATTCTGTGCGATAGCGCCCCTGTGTGTTCTCGTTATGTTGAGGAAAAAAATAA
TGGTTGCTAAGAGATTCGAACTCTTGCATCTTACGATACCTGAGTATTCCCACA
GTTAATTCTTGAAGACGAAAGGGCCTCGTGATACGCCTATTTTTATAGGTTAAT
GTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCA
TGAGACAATAACCCTGATAAATGCTTCAATAATATTGAAAAAGGAAGAGTATG
AGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTT
GGGTGCACGAGTGGGTTACATCGAACTGGATCTCAACAGCGGTAAGATCCTTG
AGAGTTTTCGCCCCGAAGAACGTTTTCCAATGATGAGCACTTTTAAAGTTCTGC
TATGTGGCGCGGTATTATCCCGTATTGACGCCGGGCAAGAGCAACTCGGTCGCC
GCATACACTATTCTCAGAATGACTTGGTTGAGTACTCACCAGTCACAGAAAAGC
ATCTTACGGATGGCATGACAGTAAGAGAATTATGCAGTGCTGCCATAACCATGA
GTGATAACACTGCGGCCAACTTACTTCTGACAACGATCGGAGGACCGAAGGAG
CTAACCGCTTTTTTGCACAACATGGGGGATCATGTAACTCGCCTTGATCGTTGG
GAACCGGAGCTGAATGAAGCCATACCAAACGACGAGCGTGACACCACGATGCC
TGTAGCAATGGCAACAACGTTGCGCAAACTATTAACTGGCGAACTACTTACTCT
AGCTTCCCGGCAACAATTAATAGACTGGATGGAGGCGGATAAAGTTGCAGGAC
CACTTCTGCGCTCGGCCCTTCCGGCTGGCTGGTTTATTGCTGATAAATCTGGAGC
CGGTGAGCGTGGGTCTCGCGGTATCATTGCAGCACTGGGGCCAGATGGTAAGC
CCTCCCGTATCGTAGTTATCTACACGACGGGGAGTCAGGCAACTATGGATGAAC
GAAATAGACAGATCGCTGAGATAGGTGCCTCACTGATTAAGCATTGGTAACTGT
CAGACCAAGTTTACTCATATATACTTTAGATTGATTTAAAACTTCATTTTTAATT
TAAAAGGATCTAGGTGAAGATCCTTTTTGATAATCTCATGACCAAAATCCCTTA
ACGTGAGTTTTCGTTCCACTGAGCGTCAGACCCCGTAGAAAAGATCAAAGGATC
TTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCG
AAGGTAACTGGCTTCAGCAGAGCGCAGATACCAAATACTGTTCTTCTAGTGTAG
CCGTAGTTAGGCCACCACTTCAAGAACTCTGTAGCACCGCCTACATACCTCGCT
CTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGA
GATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGG
AGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCC
TTTGAGTGAGCTGATACCGCTCGCCGCAGCCGAACGACCGAGCGCAGCGAGTC
AGTGAGCGAGGAAGCGGAAGAGCGCCCAA

### REFERENCES

Bernardi, A., and Spahr, P. F. (1972). Nucleotide sequence at the binding site for coat protein on RNA of bacteriophage R17. Proceedings of the National Academy of Sciences of the United States of America 69, 3033-3037.
Bertrand, E., Chartrand, P., Schaefer, M., Shenoy, S. M., Singer, R. H., and Long, R. M. (1998). Localization of ASH1 mRNA particles in living yeast. Molecular cell 2, 437-445.
Caponigro, G., Muhlrad, D., and Parker, R. (1993). A small segment of the MAT alpha 1 transcript promotes mRNA decay in Saccharomyces cerevisiae: a stimulatory role for rare codons. Molecular and cellular biology 13, 5141-5148.
Chao, J. A., Patskovsky, Y., Almo, S. C., and Singer, R. H. (2008). Structural basis for the coevolution of a viral RNA-protein complex. Nature structural & molecular biology 15, 103-105.
Eser, P., Demel, C., Maier, K. C., Schwalb, B., Pirkl, N., Martin, D. E., Cramer, P., and Tresch, A. (2014). Periodic mRNA synthesis and degradation co-operate during cell cycle gene expression. Molecular systems biology 10, 717.
Fusco, D., Accornero, N., Lavoie, B., Shenoy, S. M., Blanchard, J. M., Singer, R. H., and Bertrand, E. (2003). Single mRNA molecules demonstrate probabilistic movement in living mammalian cells. Current biology: CB 13, 161-167.
Garcia, J. F., and Parker, R. (2015). MS2 coat protein bound to yeast mRNAs block 5′ to 3′ degradation and trap mRNA decay products: implications for the localization of mRNAs by MS2-MCP system. RNA (New York, NY)
Garcia, J. F., and Parker, R. (2016). Ubiquitous accumulation of 3′ mRNA decay fragments in Saccharomyces cerevisiae mRNAs with chromosomally integrated MS2 arrays. RNA (New York, NY 22, 657-659).
Grunwald, D., and Singer, R. H. (2010). In vivo imaging of labelled endogenous beta-actin mRNA during nucleocytoplasmic transport. Nature 467, 604-607.
Guet, D., Burns, L. T., Maji, S., Boulanger, J., Hersen, P., Wente, S. R., Salamero, J., and Dargemont, C. (2015). Combining Spinach-tagged RNA and gene localization to image gene expression in live yeast. Nature communications 6, 8882.
Haim-Vilmovsky, L., Gadir, N., Herbst, R. H., and Gerst, J. E. (2011). A genomic integration method for the simultaneous visualization of endogenous mRNAs and their translation products in living yeast. RNA (New York, NY 17, 2249-2255).
Haim-Vilmovsky, L., and Gerst, J. E. (2009). m-TAG: a PCR-based genomic integration method to visualize the localization of specific endogenous mRNAs in vivo in yeast. Nature protocols 4, 1274-1284.
Haim, L., Zipor, G., Aronov, S., and Gerst, J. E. (2007). A genomic integration method to visualize localization of endogenous mRNAs in living yeast. Nature methods 4, 409-412.
Haimovich, G., Medina, D. A., Causse, S. Z., Garber, M., Millan-Zambrano, G., Barkai, O., Chavez, S., Perez-Ortin, J. E., Darzacq, X., and Choder, M. (2013). Gene expression is circular: factors for mRNA degradation also foster mRNA synthesis. Cell 153, 1000-1011.
Haimovich, G., Zabezhinsky, D., Haas, B., Slobodin, B., Purushothaman, P., Fan, L., Levin, J. Z., Nusbaum, C., and Gerst, J. E. (2016). Use of the MS2 aptamer and coat protein for RNA localization in yeast: A response to “MS2 coat proteins bound to yeast mRNAs block 5′ to 3′ degradation and trap mRNA decay products: implications for the localization of mRNAs by MS2-MCP system”. RNA (New York, NY 22, 660-666).
Heinrich, S., Sidler, C. L., Azzalin, C. M., and Weis, K. (2017). Stem-loop RNA labeling can affect nuclear and cytoplasmic mRNA processing. RNA (New York, NY 23, 134-141.
Heym, R. G., and Niessing, D. (2012). Principles of mRNA transport in yeast. Cellular and molecular life sciences: CMLS 69, 1843-1853.
Hocine, S., Raymond, P., Zenklusen, D., Chao, J. A., and Singer, R. H. (2013). Single-molecule analysis of gene expression using two-color RNA labeling in live yeast. Nature methods 10, 119-121.
Hsu, C., Scherrer, S., Buetti-Dinh, A., Ratna, P., Pizzolato, J., Jaquet, V., and Becskei, A. (2012). Stochastic signalling rewires the interaction map of a multiple feedback network during yeast evolution. Nature communications 3, 682.
Ingolia, N. T., Ghaemmaghami, S., Newman, J. R. S., and Weissman, J. S. (2009). Genome-Wide Analysis in Vivo of Translation with Nucleotide Resolution Using Ribosome Profiling. Science (New York, NY 324, 218-223).
Kshirsagar, M., and Parker, R. (2004). Identification of Edc3p as an enhancer of mRNA decapping in Saccharomyces cerevisiae. Genetics 166, 729-739.
Larson, D. R., Zenklusen, D., Wu, B., Chao, J. A., and Singer, R. H. (2011). Real-time observation of transcription initiation and elongation on an endogenous yeast gene. Science (New York, NY 332, 475-478).
Lohr, D., Venkov, P., and Zlatanova, J. (1995). Transcriptional regulation in the yeast GAL gene family a complex genetic network. FASEB journal: official publication of the Federation of American Societies for Experimental Biology 9, 777-787.
Long, R. M., Chartrand, P., Gu, W., Meng, X. H., Schaefer, M. R., and Singer, R. H. (1997a). Characterization of transport and localization of ASH1 mRNA in yeast. Molecular biology of the cell 8, 2060-2060.
Long, R. M., Singer, R. H., Meng, X., Gonzalez, I., Nasmyth, K., and Jansen, R. P. (1997b). Mating type switching in yeast controlled by asymmetric localization of ASH1 mRNA. Science (New York, NY 277, 383-387).
Lowary, P. T., and Uhlenbeck, O. C. (1987). An RNA mutation that increases the affinity of an RNA-protein interaction. Nucleic acids research 15, 10483-10493.
Mumberg, D., Muller, R., and Funk, M. (1995). Yeast vectors for the controlled expression of heterologous proteins in different genetic backgrounds. Gene 156, 119-122.
Nelles, D. A., Fang, M. Y., O'Connell, M. R., Xu, J. L., Markmiller, S. J., Doudna, J. A., and Yeo, G. W. (2016). Programmable RNA Tracking in Live Cells with CRISPR/Cas9. Cell 165, 488-496.
Pereira, G., and Schiebel, E. (2001). The role of the yeast spindle pole body and the mammalian centrosome in regulating late mitotic events. Current opinion in cell biology 13, 762-769.
Shav-Tal, Y., Darzacq, X., Shenoy, S. M., Fusco, D., Janicki, S. M., Spector, D. L., and Singer, R. H. (2004). Dynamics of single mRNPs in nuclei of living cells. Science (New York, NY 304, 1797-1800.
Sheth, U., and Parker, R. (2003). Decapping and decay of messenger RNA occur in cytoplasmic processing bodies. Science (New York, NY 300, 805-808).
Simpson, C. E., Lui, J., Kershaw, C. J., Sims, P. F., and Ashe, M. P. (2014). mRNA localization to P-bodies in yeast is bi-phasic with many mRNAs captured in a late Bfrlp-dependent wave. Journal of cell science 127, 1254-1262.
Trcek, T., Larson, D. R., Moldon, A., Query, C. C., and Singer, R. H. (2011). Single-molecule mRNA decay measurements reveal promoter-regulated mRNA stability in yeast. Cell 147, 1484-1497.
Valegard, K., Murray, J. B., Stockley, P. G., Stonehouse, N.J., and Liljas, L. (1994). Crystal structure of an RNA bacteriophage coat protein-operator complex. Nature 371, 623-626.
Valegard, K., Murray, J. B., Stonehouse, N.J., van den Worm, S., Stockley, P. G., and Liljas, L. (1997). The three-dimensional structures of two complexes between recombinant MS2 capsids and RNA operator fragments reveal sequence-specific protein-RNA interactions. Journal of molecular biology 270, 724-738.
Vera, M., Biswas, J., Senecal, A., Singer, R. H., and Park, H. Y. (2016). Single-Cell and Single-Molecule Analysis of Gene Expression Regulation. Annual review of genetics 50, 267-291.
Wu, B., Eliscovich, C., Yoon, Y. J., and Singer, R. H. (2016). Translation dynamics of single mRNAs in live cells and neurons. Science (New York, NY 352, 1430-1435.
Wu, B., Miskolci, V., Sato, H., Tutucci, E., Kenworthy, C. A., Donnelly, S. K., Yoon, Y. J., Cox, D., Singer, R. H., and Hodgson, L. (2015). Synonymous modification results in high-fidelity gene expression of repetitive protein and nucleotide sequences. Genes & development 29, 876-886.
Zenklusen, D., Larson, D. R., and Singer, R. H. (2008). Single-RNA counting reveals alternative modes of gene expression in yeast. Nature structural & molecular biology 15, 1263-1271.
Zid, B. M., and O'Shea, E. K. (2014). Promoter sequences direct cytoplasmic localization and translation of mRNAs during starvation in yeast. Nature 514, 117-121.
Zipor, G., Haim-Vilmovsky, L., Gelin-Licht, R., Gadir, N., Brocard, C., and Gerst, J. E. (2009). Localization of mRNAs coding for peroxisomal proteins in the yeast, Saccharomyces cerevisiae. Proceedings of the National Academy of Sciences of the United States of America 106, 19848-19853.
Caponigro, G., Muhlrad, D., and Parker, R. (1993). A small segment of the MAT alpha 1 transcript promotes mRNA decay in Saccharomyces cerevisiae: a stimulatory role for rare codons. Molecular and cellular biology 13, 5141-5148.
Chao, J. A., Patskovsky, Y., Almo, S. C., and Singer, R. H. (2008). Structural basis for the coevolution of a viral RNA-protein complex. Nature structural & molecular biology 15, 103-105.
Lionnet, T., Czaplinski, K., Darzacq, X., Shav-Tal, Y., Wells, A. L., Chao, J. A., Park, H. Y., de Tunis, V., Lopez-Jones, M., and Singer, R. H. (2011). A transgenic mouse for in vivo detection of endogenous labeled mRNA. Nature methods 8, 165-170.
Mueller, F., Senecal, A., Tantale, K., Marie-Nelly, H., Ly, N., Collin, O., Basyuk, E., Bertrand, E., Darzacq, X., and Zimmer, C. (2013). FISH-quant: automatic counting of transcripts in 3D FISH images. Nature methods 10, 277-278.
Passos, D. O., and Parker, R. (2008). Analysis of cytoplasmic mRNA decay in Saccharomyces cerevisiae. Methods in enzymology 448, 409-427.
Trcek, T., Chao, J. A., Larson, D. R., Park, H. Y., Zenklusen, D., Shenoy, S. M., and Singer, R. H. (2012). SinglemRNA counting using fluorescent in situ hybridization in budding yeast. Nature protocols 7, 408-419.
Wu, B., Chao, J. A., and Singer, R. H. (2012). Fluorescence fluctuation spectroscopy enables quantitative imaging of single mRNAs in living cells. Biophysical journal 102, 2936-2944.
Wu, B., Eliscovich, C., Yoon, Y. J., and Singer, R. H. (2016). Translation dynamics of single mRNAs in live cells and neurons. Science (New York, NY 352, 1430-1435).

1.-16. (canceled)

17. A method of detecting or of monitoring an RNA of interest in a cell, the method comprising:
(i) inserting in a cell a nucleic acid encoding from seven to twenty four loops of 5′-ANC/UA-3′ into the 3′-UTR of a gene encoding the RNA of interest by Cre-Lox recombination so as to replace a kanamycin resistance gene with the RNA of interest;
(ii) transfecting the cell with a nucleic acid encoding a MS2 bacteriophage coat protein homo-dimer (MCP) so as to permit expression of a fusion protein comprising the MCP, a nuclear localization sequence (NLS) and a first and a second fluorescent protein; and
(iii) detecting or monitoring the movement and/or location of a fluorescent signal of the first and second fluorescent proteins so as to detect or monitor the RNA of interest.

18. The method of claim 17, wherein the cell is a yeast cell.

19. The method of claim 17, wherein in (i) the nucleic acid comprises SEQ ID NO:2 or 3.

20. The method of claim 17, wherein in (ii) the nucleic acid comprises SEQ ID NO:4 or 32.

21.-27. (canceled)

28. The method of claim 17, wherein in (i) the nucleic acid encodes from twelve to twenty four loops of 5′-ANC/UA-3′.

29. The method of claim 17, wherein in (i) the nucleic acid encodes from seven to twelve loops of 5′-ANC/UA-3′.

30. The method of claim 17, wherein in (i) the 5′ end of each loop is connected to a sequence of eight nucleotides, seven of which are complementary to a sequence of seven nucleotides connected to the 3′ end of the same loop, such that a stem and loop structure is formed for each loop, and wherein a stem of each loop is separated from a stem of each adjacent loop by a nucleotide sequence of 40-55 nucleotides.

31. The method of claim 17, wherein the 5′ end of each loop is connected to a sequence of eight nucleotides, seven of which are complementary to a sequence of seven nucleotides connected to the 3′ end of the same loop, such that a stem and loop structure is formed for each loop, and wherein a stem of each loop is separated from a stem of each adjacent loop by a nucleotide sequence of 45-55 nucleotides.

32. The method of claim 17, wherein in (i) the nucleic acid, at a 3′ portion thereof, further comprises a two LoxP sites, optionally separated by a marker gene.

33. The method of claim 17, wherein in (i) the nucleic acid encodes twelve loops of 5′-ANC/UA-3′.

34. The method of claim 17, wherein each loop is separated from each adjacent loop by a nucleotide sequence of 50 nucleotides.

35. The method of claim 17, wherein the stem of the stem and loop structure for each of the loops has a different sequence than the stems of the stem and loop structure for all of the remaining loops.

36. The method of claim 17, wherein the stem of the stem and loop structure for each of the loops has the same sequence as the stems of the stem and loop structure for all of the remaining loops.

37. The method of claim 17, wherein in (i) the loops encoded by the nucleic acid all have the sequence 5′-ANUA-3′.

38. The method of claim 17, wherein in (i) the loops encoded by the nucleic acid all have the sequence 5′-ANCA-3′.

39. The method of claim 17, wherein in (ii) the nucleic acid encodes, in 5′ to 3′ order or 3′ to 5′ order:
(i) a CYC1 promoter;
(ii) a MS2 bacteriophage coat protein homo-dimer (MCP);
(iii) a first fluorescent protein;
(iv) a second fluorescent protein;
(v) a nuclear localization sequence (NLS);
(vi) a CYC1 terminator sequence.

40. The method of claim 17, wherein in (ii) the nucleic acid encodes, in 5′ to 3′ order or 3′ to 5′ order:
(i) a CYC1 promoter;
(ii) a MS2 bacteriophage coat protein homo-dimer (MCP);
(iii) a nuclear localization sequence (NLS);
(iv) a first fluorescent protein;
(v) a second fluorescent protein;
(vi) a CYC1 terminator sequence.

41. The method of claim 39, wherein the nucleic acid encodes (i) through (iv) in 5′ to 3′ order.

42. The method of claim of claim 39, wherein the first fluorescent protein and second fluorescent protein have the same amino acid sequence.

43. The method of claim 39, wherein the first fluorescent protein and second fluorescent protein are a GFP and/or a tdTomato.

44. The method of claim 39, wherein the first fluorescent protein and second fluorescent protein are eGFP.

45. The method of claim 40, wherein the nucleic acid encodes (i) through (iv) in 5′ to 3′ order.

46. The method of claim of claim 40, wherein the first fluorescent protein and second fluorescent protein have the same amino acid sequence.

47. The method of claim 40, wherein the first fluorescent protein and second fluorescent protein are a GFP and/or a tdTomato.

48. The method of claim 40, wherein the first fluorescent protein and second fluorescent protein are eGFP.


SEQUENCE LISTING (39 sequences)

SEQ ID NO: 1 - skipped

SEQ ID NO: 2 - DNA, 5330 bp, unidentified
  source 1..5330: mol_type = unassigned DNA; note = Plasmid sequence
tattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccagagccccctggcaatcgcggggagacgcaggactaccgcgtcttcactctcgcttgcgcggtatacgcagggagcaacgtcacccagggcgacacagaggaataccctgtgtggccccacggtgctacaaagaacattccgtattgctcaccagtgtcacatgtggaggactaccccacaagcgagtcaggaaaccttcggggcatcgcaccattatcccgaacaatcgacacgcaggattaccgcgtgggacactctgttccccgtcaaaattggaccataccggagtcggcttacgtcatgcaggattaccgcatgcatggtgcagaatatcggcatgtctactcgtacagtcaaatctactcgtgtggtcaggactaccgaccacttctattctattcatcttttcggttgtgcaggtattctgccgatgtacgagaagacgattacgcttctcgactaccttcatcatacatggtgtgcagatggccgccaagttttttgccaatggaggaataccccattctctgtcaaccaacccatgcaaagtttacactctgctatggcagcactgtcgcagaggaataccctgcgagccaaaacggcccccggtgcgtgtattgcatctgccttgcgagcattcacagggacgaatacgccctgccgttgcattacttcaatatgggtgctctgtcgtcgtcatcaggaccatttgcgcaggactaccgcgcatatatcatcagcactcgtgcggatacttctgggattcctattgttacgcgagctcaggaataccgagctctggcgacagagaccctcacacggaagatctatcgatctcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactagtggcctatgcggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtc

SEQ ID NO: 3 - DNA, 5330 bp, unidentified
  source 1..5330: mol_type = unassigned DNA; note = Plasmid sequence
tattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccagagccccctggcaatcgcggggagacgcaggaccaccgcgtcttcactctcgcttgcgcggtatacgcagggagcaacgtcacccagggcgacacagaggaacaccctgtgtggccccacggtgctacaaagaacattccgtattgctcaccagtgtcacatgtggaggaccaccccacaagcgagtcaggaaaccttcggggcatcgcaccattatcccgaacaatcgacacgcaggatcaccgcgtgggacactctgttccccgtcaaaattggaccataccggagtcggcttacgtcatgcaggatcaccgcatgcatggtgcagaatatcggcatgtctactcgtacagtcaaatctactcgtgtggtcaggaccaccgaccacttctattctattcatcttttcggttgtgcaggtattctgccgatgtacgagaagacgatcacgcttctcgactaccttcatcatacatggtgtgcagatggccgccaagttttttgccaatggaggaacaccccattctctgtcaaccaacccatgcaaagtttacactctgctatggcagcactgtcgcagaggaacaccctgcgagccaaaacggcccccggtgcgtgtattgcatctgccttgcgagcattcacagggacgaacacgccctgccgttgcattacttcaatatgggtgctctgtcgtcgtcatcaggaccatttgcgcaggaccaccgcgcatatatcatcagcactcgtgcggatacttctgggattcctattgttacgcgagctcaggaacaccgagctctggcgacagagaccctcacacggaagatctatcgatctcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactagtggcctatgcggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtc

SEQ ID NO: 4 - DNA, 8469 bp, unidentified
  source 1..8469: mol_type = unassigned DNA; note = Plasmid sequence
tacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagggagcgttggttggtggatcaagcccacgcgtaggcaatcctcgagcagatccgccgggcgtgtatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggtcctttgtagcataaattactatacttctatagacacgcaaacacaaatacacacactaaattaataggatccatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacccaaaaaaaaaaagaaaagttaccggttctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcaaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaatgtttaaactctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataagtttaaacccgctgatcctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcgctgggccattctcatgaagaatatcttgaatttattgtcatattactagttggtgtggaagtcctaatatcggtgatcaatatagtggttgacatgctggctagtcaacattgagccttttgatcatgcaaatatattacggtattttacaatcaaatatcaaacttaactattgactttataacttatttaggtggtaacattcttataaaaaagaaaaaaattactgcaaaacagtactagcttttaacttgtatcctaggttatctatgctgtctcaccatagagaatattacctatttcagaatgtatgtccatgattcgccgggtaaatacatataatacacaaatctggcttaataaagtctataatatatctcataaagaagtgctaaattggctagtgctatatatttttaagaaaatttcttttgactaagtccatatcgactttgtaaaagttcacattagcatacatatattacacgagccagaaatagtaacttttgcctaaatcacaaattgcaaaatttaattgcttgcaaaaggtcacatgcttataatcaacttttttaaaaatttaaaatacttttttattttttatttttaaacataaatgaaataatttatttattgtttatgattaccgaaacataaaacctgctcaagaaaaagaaactgttttgtccttggaaaaaaagcactacctaggagcggccaaaatgccgaggctttcatagcttaaactctttacagaaaataggcattatagatcagttcgagttttcttattcttccttccggttttatcgtcacagttttacagtaaataagtatcacctcttagagttcgatgataagctgtcaaacatgagaattaattccacatgttaaaatagtgaaggagcatgttcggcacacagtggaccgaacgtggggtaagtgcactagggtccggttaaacggatctcgcattgatgaggcaacgctaattatcaacatatagattgttatctatctgcatgaacacgaaatctttacttgacgacttgaggctgatggtgtttatgcaaagaaaccactgtgtttaatatgtgtcactgtttgatattactgtcagcgtagaagataatagtaaaagcggttaataagtgtatttgagataagtgtgataaagtttttacagcgaaaagacgataaatacaagaaaatgattacgaggatacggagagaggtatgtacatgtgtatttatatactaagctgccggcggttgtttgcaagaccgagaaaaggctagcaagaatcgggtcattgtagcgtatgcgcctgtgaacattctcttcaacaagtttgattccattgcggtgaaatggtaaaagtcaaccccctgcgatgtatattttcctgtacaatcaatcaaaaagccaaatgatttagcattatctttacatcttgttattttacagattttatgtttagatcttttatgcttgcttttcaaaaggcttgcaggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatcgatgataagctgtcaaacatgagaattaattctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaa

SEQ ID NO: 5 - DNA, 19 bp, unidentified
  source 1..19: mol_type = unassigned DNA; note = Bacteriophage MS2 sequence
acatgaggattacccatgt

SEQ ID NO: 6 - DNA, 19 bp, unidentified
  source 1..19: mol_type = unassigned DNA; note = Bacteriophage MS2 sequence
acatgaggatcacccatgt

SEQ ID NO: 7 - DNA, 19 bp, synthetic construct
  source 1..19: mol_type = other DNA; note = Synthetic variant based on Bacteriophage MS2 sequence
gacgcaggactaccgcgtc

SEQ ID NO: 8 - DNA, 19 bp, synthetic construct
  source 1..19: mol_type = other DNA; note = Synthetic variant based on Bacteriophage MS2 sequence
gacgcaggaccaccgcgtc

SEQ ID NO: 9 - DNA, 19 bp, synthetic construct
  source 1..19: mol_type = other DNA; note = Synthetic variant based on Bacteriophage MS2 sequence
cgcagaggaataccctgcg

SEQ ID NO: 10 - DNA, 19 bp, synthetic construct
  source 1..19: mol_type = other DNA; note = Synthetic variant based on Bacteriophage MS2 sequence
cgcagaggaacaccctgcg

SEQ ID NO: 11 - DNA, 50 bp, Drosophila sp.
  source 1..50: mol_type = genomic DNA
acaaggagagaaatgtacaattgtttcgtgataatgtctcttattagttg

SEQ ID NO: 12 - DNA, 18 bp, synthetic construct
  source 1..18: mol_type = other DNA; note = Synthetic ORP100 PROBE sequence
gtctagccgcgaggaagg

SEQ ID NO: 13 - DNA, 87 bp, Drosophila sp.
  source 1..87: mol_type = genomic DNA
tgcgaaattgaagggtaccgttgcttattttgtaattacataactgagacagtagagaattgaaacctacaaacgggtggaggatca

SEQ ID NO: 14 - DNA, 83 bp, Drosophila sp.
  source 1..83: mol_type = genomic DNA
tgcgaaattgaagggtaccgttgcttattttgtaattacataactgagacagtagagaattgaccgctctagaactagtggat

SEQ ID NO: 15 - DNA, 82 bp, Drosophila sp.
  source 1..82: mol_type = genomic DNA
tgtacaattgtttcgtgataatgtctcttattagttgaaagagattcagttatccatgtagcataggccactagtggatctg

SEQ ID NO: 16 - DNA, 23 bp, Drosophila sp.
  source 1..23: mol_type = genomic DNA
aacacatacaagatgtttgaacg

SEQ ID NO: 17 - DNA, 21 bp, Brucella abortus
  source 1..21: mol_type = genomic DNA
ctgattgcccgacattatcgc

SEQ ID NO: 18 - DNA, 90 bp, unidentified
  source 1..90: mol_type = unassigned DNA; note = Yeast sequence
gcttgcaaacatcaaaaggagctatgggaacgtgccaaggtttaaggatattttcgacgatctctcctaaccgctctagaactagtggat

SEQ ID NO: 19 - DNA, 92 bp, unidentified
  source 1..92: mol_type = unassigned DNA; note = Yeast sequence
gggcagaaagaattttaaagattatttgctatctagacattatgtgttttatatgattgctgtaaaagtagcataggccactagtggatctg

SEQ ID NO: 20 - DNA, 20 bp, unidentified
  source 1..20: mol_type = unassigned DNA; note = Yeast sequence
ggaacgtgccaaggtttaag

SEQ ID NO: 21 - DNA, 95 bp, Homo sapiens
  source 1..95: mol_type = genomic DNA
cacgatataagcgaactacccgaaatgctttcactgattttgcgtcaatactttacagacctggcatccagctaacgctctagaactagtggatc

SEQ ID NO: 22 - DNA, 98 bp, Homo sapiens
  source 1..98: mol_type = genomic DNA
ttttttttcagttccatgatttttttgttcctttgattcgtgtagtaaacctcctcttcttggttttcacgatatacactagtggatctgatatcacc

SEQ ID NO: 23 - DNA, 69 bp, Homo sapiens
  source 1..69: mol_type = genomic DNA
ttcactgattttgcgtcaatactttacagacctggcatccagctaagccgctctagaactagtggatcc

SEQ ID NO: 24 - DNA, 69 bp, Homo sapiens
  source 1..69: mol_type = genomic DNA
cctttgattcgtgtagtaaacctcctcttcttggttttcacgatatagcataggccactagtggatctg

SEQ ID NO: 25 - DNA, 21 bp, Brucella abortus
  source 1..21: mol_type = genomic DNA
ctgattgcccgacattatcgc

SEQ ID NO: 26 - DNA, 22 bp, Homo sapiens
  source 1..22: mol_type = genomic DNA
gatggtatttgcgaagaccatg

SEQ ID NO: 27 - DNA, 85 bp, unidentified
  source 1..85: mol_type = unassigned DNA; note = Yeast sequence
tgctgagctagaaaatgctatcatcgtctctaaaccagcattgggcagctgtctatatgaattataagcccctggcaatcgcggg

SEQ ID NO: 28 - DNA, 94 bp, unidentified
  source 1..94: mol_type = unassigned DNA; note = Yeast sequence
ttttgtgatgctaaagttatgagtagaaaaaaatgagaagttgttctgaacaaagtaaaaaaaagaagtatacactagtggatctgatatcacc

SEQ ID NO: 29 - DNA, 94 bp, unidentified; identical to SEQ ID NO: 28
  source 1..94: mol_type = unassigned DNA; note = Yeast sequence

SEQ ID NO: 30 - DNA, 21 bp, unidentified
  source 1..21: mol_type = unassigned DNA; note = Yeast sequence
gaagcccttgccaatgagttc

SEQ ID NO: 31 - DNA, 28 bp, synthetic construct
  source 1..28: mol_type = other DNA; note = probe based on Drosophila
gtttcgtgataatgtctcttattagttg

SEQ ID NO: 32 - DNA, 8567 bp, unidentified
  source 1..8567: mol_type = unassigned DNA; note = Plasmid sequence
tacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagggagcgttggttggtggatcaagcccacgcgtaggcaatcctcgagcagatccgccgggcgtgtatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggtcctttgtagcataaattactatacttctatagacacgcaaacacaaatacacacactaaattaataggatccatgctagccgttaaaatggcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacaccggttctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcaaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaatgtttaaactctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaatacccaaaaaaaaaaagaaaagtttaagtttaaacccgctgatcctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattggcgcgtgtacgcatgtaacattatactgaaaaccttgcttgagaaggttttgggacgctcgaaggctttaatttgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcataatcgctgggccattctcatgaagaatatcttgaatttattgtcatattactagttggtgtggaagtcctaatatcggtgatcaatatagtggttgacatgctggctagtcaacattgagccttttgatcatgcaaatatattacggtattttacaatcaaatatcaaacttaactattgactttataacttatttaggtggtaacattcttataaaaaagaaaaaaattactgcaaaacagtactagcttttaacttgtatcctaggttatctatgctgtctcaccatagagaatattacctatttcagaatgtatgtccatgattcgccgggtaaatacatataatacacaaatctggcttaataaagtctataatatatctcataaagaagtgctaaattggctagtgctatatatttttaagaaaatttcttttgactaagtccatatcgactttgtaaaagttcacattagcatacatatattacacgagccagaaatagtaacttttgcctaaatcacaaattgcaaaatttaattgcttgcaaaaggtcacatgcttataatcaacttttttaaaaatttaaaatacttttttattttttatttttaaacataaatgaaataatttatttattgtttatgattaccgaaacataaaacctgctcaagaaaaagaaactgttttgtccttggaaaaaaagcactacctaggagcggccaaaatgccgaggctttcatagcttaaactctttacagaaaataggcattatagatcagttcgagttttcttattcttccttccggttttatcgtcacagttttacagtaaataagtatcacctcttagagttcgatgataagctgtcaaacatgagaattaattccacatgttaaaatagtgaaggagcatgttcggcacacagtggaccgaacgtggggtaagtgcactagggtccggttaaacggatctcgcattgatgaggcaacgctaattatcaacatatagattgttatctatctgcatgaacacgaaatctttacttgacgacttgaggctgatggtgtttatgcaaagaaaccactgtgtttaatatgtgtcactgtttgatattactgtcagcgtagaagataatagtaaaagcggttaataagtgtatttgagataagtgtgataaagtttttacagcgaaaagacgataaatacaagaaaatgattacgaggatacggagagaggtatgtacatgtgtatttatatactaagctgccggcggttgtttgcaagaccgagaaaaggctagcaagaatcgggtcattgtagcgtatgcgcctgtgaacattctcttcaacaagtttgattccattgcggtgaaatggtaaaagtcaaccccctgcgatgtatattttcctgtacaatcaatcaaaaagccaaatgatttagcattatctttacatcttgttattttacagattttatgtttagatcttttatgcttgcttttcaaaaggcttgcaggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatcgatgataagctgtcaaacatgagaattaattctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaa

SEQ ID NO: 33 - RNA, 19 nt, synthetic construct
  source 1..19: mol_type = other RNA; note = Synthetic artificial sequence based on phage genome
acatcaccattacccatct

SEQ ID NO: 34 - RNA, 19 nt, synthetic construct
  source 1..19: mol_type = other RNA; note = Synthetic artificial sequence based on phage genome
acatgaggatcacccatgt

SEQ ID NO: 35 - RNA, 19 nt, synthetic construct
  source 1..19: mol_type = other RNA; note = Synthetic artificial sequence based on phage genome
acatgaggattacccatgt

SEQ ID NO: 36 - RNA, 19 nt, synthetic construct
  source 1..19: mol_type = other RNA; note = Synthetic artificial sequence based on phage genome
gacgcaggaccaccgcgtc

SEQ ID NO: 37 - RNA, 19 nt, synthetic construct
  source 1..19: mol_type = other RNA; note = Synthetic artificial sequence based on phage genome
gacgcaggactaccgcgtc

SEQ ID NO: 38 - RNA, 19 nt, synthetic construct
  source 1..19: mol_type = other RNA; note = Synthetic artificial sequence based on phage genome
cgcagaggaacaccctgcg

SEQ ID NO: 39 - RNA, 19 nt, synthetic construct
  source 1..19: mol_type = other RNA; note = Synthetic artificial sequence based on phage genome
cgcagaggaataccctgcg